SEQUENCE LISTING (47 sequences)

SEQ ID NO: 1 - protein, 20 aa, Artificial
  Phl p 1 epitope sequence
EEPIAPYHFDLSGHAFGAMA

SEQ ID NO: 2 - protein, 20 aa, Artificial
  Phl p 1 epitope sequence
TEAEDVIPEGWKADTSYESK

SEQ ID NO: 3 - protein, 20 aa, Artificial
  Phl p 1 epitope sequence
WYGKPTGAGPKDNGGACGYK

SEQ ID NO: 4 - protein, 20 aa, Artificial
  Phl p 5a epitope sequence
LDAAYKLAYKTAEGATPEAK

SEQ ID NO: 5 - protein, 15 aa, Artificial
  Phl p 5b epitope sequence
DTYKCIPSLEAAVKQ

SEQ ID NO: 6 - protein, 263 aa, Artificial
  Phl p 1 antigen sequence
MASSSSVLLVVVLFAVFLGSAYGIPKVPPGPNITATYGDKWLDAKSTWYGKPTGAGPKDNGGACGYKDVDKPPFSGMTGCGNTPIFKSGRGCGSCFEIKCTKPEACSGEPVVVHITDDNEEPIAPYHFDLSGHAFGAMAKKGDEQKLRSAGELELQFRRVKCKYPEGTKVTFHVEKGSNPNYLALLVKYVNGDGDVVAVDIKEKGKDKWIELKESWGAIWRIDTPDKLTGPFTVRYTTEGGTKTEAEDVIPEGWKADTSYESK

SEQ ID NO: 7 - protein, 312 aa, Artificial
  Phl p 5a antigen sequence
MAVHQYTVALFLAVALVAGPAASYAADLGYGPATPAAPAAGYTPATPAAPAEAAPAGKATTEEQKLIEKINAGFKAALAAAAGVQPADKYRTFVATFGAASNKAFAEGLSGEPKGAAESSSKAALTSKLDAAYKLAYKTAEGATPEAKYDAYVATLSEALRIIAGTLEVHAVKPAAEEVKVIPAGELQVIEKVDAAFKVAATAANAAPANDKFTVFEAAFNDAIKASTGGAYESYKFIPALEAAVKQAYAATVATAPEVKYTVFETALKKAITAMSEAQKAAKPAAAATATATAAVGAATGAATAATGGYKV

SEQ ID NO: 8 - protein, 284 aa, Artificial
  Phl p 5b antigen sequence
AAAAVPRRGPRGGPGRSYTADAGYAPATPAAAGAAAGKATTEEQKLIEDINVGFKAAVAAAASVPAADKFKTFEAAFTSSSKAAAAKAPGLVPKLDAAYSVAYKAAVGATPEAKFDSFVASLTEALRVIAGALEVHAVKPVTEEPGMAKIPAGELQIIDKIDAAFKVAATAAATAPADDKFTVFEAAFNKAIKESTGGAYDTYKCIPSLEAAVKQAYAATVAAAPQVKYAVFEAALTKAITAMSEVQKVSQPATGAATVAAGAATTAAGAASGAATVAAGGYKV

SEQ ID NO: 9 - protein, 13 aa, Artificial
  Aln g 1 epitope sequence
DRVNFKYSFSVIE

SEQ ID NO: 10 - protein, 13 aa, Artificial
  Aln g 1 epitope sequence
NFKYSFSVIEGGA

SEQ ID NO: 11 - protein, 13 aa, Artificial
  Aln g 1 epitope sequence
GSILKISNKFHTK

SEQ ID NO: 12 - protein, 13 aa, Artificial
  Aln g 1 epitope sequence
VGLLKAVESYLLA

SEQ ID NO: 13 - protein, 13 aa, Artificial
  Aln g 1 epitope sequence
LKAVESYLLAHSD

SEQ ID NO: 14 - protein, 13 aa, Artificial
  Aln g 1 epitope sequence
VESYLLAHSDAYN

SEQ ID NO: 15 - protein, 160 aa, Artificial
  Aln g 1 antigen sequence
MGVFNYEAETPSVIPAARLFKAFILDGDKLLPKVAPEAVSSVENIEGNGGPGTIKKITFPEGSPFKYVKERVDEVDRVNFKYSFSVIEGGAVGDALEKVCNEIKIVAAPDGGSILKISNKFHTKGDHEINAEQIKIEKEKAVGLLKAVESYLLAHSDAYN

SEQ ID NO: 16 - protein, 20 aa, Artificial
  Fel d 1 T-cell epitope sequence
VAQYKALPVVLENARILKNC

SEQ ID NO: 17 - protein, 10 aa, Artificial
  Fel d 1 T-cell epitope sequence
ELLLDLSLTK

SEQ ID NO: 18 - protein, 10 aa, Artificial
  Fel d 1 T-cell epitope sequence
LSLLDKIYTS

SEQ ID NO: 19 - protein, 13 aa, Artificial
  Fel d 1 T-cell epitope sequence
LPVVLENARILKN

SEQ ID NO: 20 - protein, 13 aa, Artificial
  Fel d 1 T-cell epitope sequence
ENALSLLDKIYTS

SEQ ID NO: 21 - protein, 13 aa, Artificial
  Fel d 1 T-cell epitope sequence
DEYVEQVAQYKAL

SEQ ID NO: 22 - protein, 20 aa, Artificial
  Phl p 1 T-cell epitope sequence
KGSNPNYLALLVKYVNGDGD

SEQ ID NO: 23 - protein, 20 aa, Artificial
  Phl p 5b T-cell epitope sequence
KLIEDINVGFKAAVAAAASV

SEQ ID NO: 24 - protein, 20 aa, Artificial
  Phl p 1 T-cell epitope sequence
GDGDVVAVDIKEKGKDKWIE

SEQ ID NO: 25 - protein, 20 aa, Artificial
  Phl p 5a T-cell sequence
PEAKYDAYVATLSEALRIIA

SEQ ID NO: 26 - protein, 20 aa, Artificial
  Phl p 5b T-cell sequence
ATPEAKFDSFVASLTEALRV

SEQ ID NO: 27 - protein, 20 aa, Artificial
  Phl p 5a T-cell epitope sequence
FAEGLSGEPKGAAESSSKAA

SEQ ID NO: 28 - protein, 20 aa, Artificial
  Ara h 1 T-cell epitope sequence
QRSRQFQNLQNHRIVQIEAK

SEQ ID NO: 29 - protein, 20 aa, Artificial
  Ara h 1 T-cell epitope sequence
DNILVIQQGQATVTVANGNN

SEQ ID NO: 30 - protein, 20 aa, Artificial
  Ara h 1 T-cell epitope sequence
KEGDVFIMPAAHPVAINASS

SEQ ID NO: 31 - protein, 20 aa, Artificial
  Ara h 1 T-cell epitope sequence
NNFGKLFEVKPDKKNPQLQD

SEQ ID NO: 32 - protein, 20 aa, Artificial
  Ara h 1 T-cell epitope sequence
QRSRQFQNLQNHRIVQIEAK

SEQ ID NO: 33 - protein, 20 aa, Artificial
  Ara h 1 T-cell epitope sequence
FNEIRRVLLEENAGGEQEER

SEQ ID NO: 34 - protein, 20 aa, Artificial
  Ara h 1 T-cell epitope sequence
KEGDVFIMPAAHPVAINASS

SEQ ID NO: 35 - protein, 20 aa, Artificial
  Ara h 1 T-cell epitope sequence
QKESHFVSARPQSQSQSPSS

SEQ ID NO: 36 - protein, 20 aa, Artificial
  Ara h 1 T-cell epitope sequence
FNEIRRVLLEENAGGEQEER

SEQ ID NO: 37 - protein, 20 aa, Artificial
  Ara h 1 T-cell epitope sequence
NSKAMVIVVVNKGTGNLELV

SEQ ID NO: 38 - protein, 20 aa, Artificial
  Ara h 1 T-cell epitope sequence
TSRNNPFYFPSRRFSTRYGN

SEQ ID NO: 39 - protein, 20 aa, Artificial
  Ara h 1 T-cell epitope sequence
LEAAFNAEFNEIRRVLLEEN

SEQ ID NO: 40 - protein, 20 aa, Artificial
  Ara h 1 T-cell epitope sequence
VVNKGTGNLELVAVRKEQQQ

SEQ ID NO: 41 - protein, 20 aa, Artificial
  Ara h 1 T-cell epitope sequence
LELVAVRKEQQQRGRREEEE

SEQ ID NO: 42 - protein, 20 aa, Artificial
  Ara h 1 T-cell epitope sequence
LEAAFNAEFNEIRRVLLEEN

SEQ ID NO: 43 - protein, 20 aa, Artificial
  Ara h 1 T-cell epitope sequence
FNEIRRVLLEENAGGEQEER

SEQ ID NO: 44 - protein, 20 aa, Artificial
  Ara h 1 T-cell epitope sequence
QRSRQFQNLQNHRIVQIEAK

SEQ ID NO: 45 - protein, 20 aa, Artificial
  Ara h 1 T-cell epitope sequence
LQNHRIVQIEAKPNTLVLPK

SEQ ID NO: 46 - protein, 20 aa, Artificial
  Ara h 1 T-cell epitope sequence
SKEHVEELTKHAKSVSKKGS

SEQ ID NO: 47 - protein, 20 aa, Artificial
  Ara h 1 T-cell epitope sequence
EEEGSNREVRRYTARLKEGD

(19) United States
(12) Patent Application Publication
(10) Pub. No.: US 2013/0004528 A1
(43) Pub. Date: Jan. 3, 2013

(54) DIRECT ANALYSIS OF ANTIGEN-SPECIFIC IMMUNE RESPONSE

(75) Inventors: William W. Kwok, Bellevue, WA (US); Erik Wambre, Seattle, WA (US)

(73) Assignee: Benaroya Research Institute, Seattle, WA (US)

(21) Appl. No.: 13/581,033

(22) PCT Filed: Feb. 25, 2011
(86) PCT No.: PCT/US2011/026244

§ 371 Date: Aug. 24, 2012

Related U.S. Application Data
(60) Provisional application No. 61/308,730, filed on Feb. 26, 2010.

(51) Int. Cl. A61K 39/00; G01N 33/566; A61P 37/08; C07K 7/08; C07K 7/06; C12Q 1/68; C40B 30/04

(52) U.S. Cl. 424/185.1; 435/6.11; 435/7.24; 506/9; 530/326; 530/327; 530/328

(57) ABSTRACT

Provided herein are methods for the determination of antigen-specific CD4+ T cell phenotype and/or frequency, which is useful for detecting or monitoring immune function, directing immunotherapy to the use of those epitopes or antigen fragments that elicit an allergic reaction (e.g., as measured by detection of a Th2 response) and/or promote immune deviation, monitoring an immune response to a particular antigen, etc.

### RELATED APPLICATIONS

This application claims the benefit under 35 U.S.C. §119(e) of U.S. Provisional Patent Application Ser. No. 61/308,730, filed Feb. 26, 2010, the disclosure of which is incorporated herein by reference in its entirety.

### STATEMENT OF GOVERNMENT SUPPORT

This invention was made with Government support under contract number HHSN272200700046C awarded by the National Institutes of Health. The Government has certain rights in the invention.

### FIELD OF THE INVENTION

The present invention is in the field of immunology and immunology-based medical treatment and/or monitoring.

### BACKGROUND

Specific immunotherapy is an approach to combat IgE-mediated allergic diseases in which gradually increasing doses of crude extracts of specific allergens is administered to a subject to build up a tolerance to the allergen. Successful immunotherapy encourages allergen-specific B cells to switch their antibody class from IgE, which is associated with allergy, asthma and anaphylactic shock, to one of the other antibody classes.

Though promising, serious side effects may occur using this method, and specific immunotherapy is not always successful. Better approaches are needed to more precisely direct the treatment of allergic disorders with immunotherapy.

### SUMMARY

The present application provides an improved approach to immunotherapy by specifically directing the immune cell exposures to epitopes that elicit a CD4+ Th2 response in a subject, preferably without the need to administer a crude allergen preparation that may elicit a IgE response.

Thus, provided herein are methods of treatment for an allergic disorder (e.g., seasonal rhinoconjuctivitis, animal dander allergy, food allergy, or venom anaphylaxis) in a subject (e.g., a human subject) in need thereof, including administering one or more Th2 eliciting polypeptides to the subject, to thereby treat the subject for the allergic disorder.

In some embodiments, the allergic disorder is an allergy to timothy grass pollen and the polypeptides are selected from the group consisting of: SEQ ID NO:1, SEQ ID NO:2, SEQ ID NO:3, SEQ ID NO:4, SEQ ID NO:5, SEQ ID NO:22, SEQ ID NO:23, SEQ ID NO:24, SEQ ID NO:25, SEQ ID NO:26, SEQ ID NO:27, and homologues thereof.

In some embodiments, the allergic disorder is an allergy to alder pollen and the polypeptides are selected from the group consisting of: SEQ ID NO:9, SEQ ID NO:10, SEQ ID NO:11, SEQ ID NO:12, SEQ ID NO:13, SEQ ID NO:14, and homologues thereof.

In some embodiments, the allergic disorder is a feline allergy and the polypeptides are selected from the group consisting of: SEQ ID NO:16, SEQ ID NO:17, SEQ ID NO:18, SEQ ID NO:19, SEQ ID NO:20, SEQ ID NO:21, and homologues thereof.

In some embodiments, the allergic disorder is a peanut allergy and the polypeptides are selected from the group consisting of: SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, and homologues thereof.

In some embodiments, the methods further include determining the MHC class II genotype of the subject (e.g., by nucleic acid amplification). In some embodiments, the polypeptides administered are selected based upon the MHC class II genotype of the subject, which may allow for an even greater refinement of the peptides used for immunotherapy.

Also provided are compositions comprising, consisting of, or consisting essentially of polypeptides which elicit a Th2 CD4+ T cell response (e.g., in a subject with a predetermined MHC class II molecule genotype). In some embodiments, the composition includes: (a) a polypeptide selected from the group consisting of: SEQ ID NO:1, SEQ ID NO:2, SEQ ID NO:3 SEQ ID NO:4, SEQ ID NO:5, SEQ ID NO:22, SEQ ID NO:23, SEQ ID NO:24, SEQ ID NO:25, SEQ ID NO:26, SEQ ID NO:27, and homologues thereof; (b) a polypeptide selected from the group consisting of: SEQ ID NO:9, SEQ ID NO:10, SEQ ID NO:11, SEQ ID NO:12, SEQ ID NO:13, SEQ ID NO:14, and homologues thereof; (c) a polypeptide selected from the group consisting of: SEQ ID NO:16, SEQ ID NO:17, SEQ ID NO:18, SEQ ID NO:19, SEQ ID NO:20, SEQ ID NO:21, and homologues thereof; and/or (d) a polypeptide selected from the group consisting of: SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, and homologues thereof.

In some embodiments, the compositions further include a carrier. In some embodiments, the composition is formulated for subcutaneous or sublingual administration.

Further provided are methods for determining a CD4+ T cell response to a predetermined complex, the complex including a predetermined MHC class II molecule and a predetermined epitope that binds to the predetermined MHC class II molecule, including: contacting the CD4+ T cells with the predetermined complex; and determining whether the response of the CD4+ T cells is a Th1 or a Th2 response; to thereby determine the type of CD4+ T cell response to the complex. In some embodiments, the determining comprises detecting cytokine secretion and/or cell surface markers of the CD4+ T cells. In some embodiments, the predetermined complex comprises MHC class II tetramers.

Methods for determining whether a subject is at risk for an allergic reaction to an allergen of interest are also provided, the method including: determining the MHC class II genotype of the subject; and then determining whether the subject is at risk for an allergic reaction to the allergen of interest based upon the MHC class II genotype.

Further provided are methods for testing or monitoring a CD4+ T cell response in a subject (e.g., subject is afflicted with an allergic disease, an autoimmune disease (e.g., multiple sclerosis, diabetes type I), or a cancer) to an antigen, the antigen including a predetermined epitope, the method including: obtaining a blood sample from the subject, the blood sample including CD4+ T cells; providing a complex including the predetermined epitope and a predetermined MHC class II molecule that binds to the predetermined epitope; contacting the CD4+ T cells with the complex; and determining whether the response of the CD4+ T cells is a Th1 or a Th2 response; to thereby determine the type of CD4+ T cell response to the antigen. In some embodiments, the determining comprises detecting cytokine secretion and/or cell surface markers of the CD4+ T cells. In some embodiments, the predetermined complex comprises MHC class II tetramers.

In some embodiments, the blood sample is a whole blood sample sample or a peripheral blood mononuclear cell (PBMC) sample. In some embodiments, the sample has a volume of from 0.1 to 10 milliliters, or from 0.5 to 5 milliliters.

In some embodiments, the antigen is a vaccine antigen.

Also provided is the use of one or more polypeptides as provided herein for the treatment of an allergic disorder, or in the preparation of a medicament for the treatment of an allergic disorder.

### BRIEF DESCRIPTION OF THE DRAWINGS

FIG. 1A-1B. Flow diagrams of assay methods according to some embodiments.

FIG. 2. Exemplary Dot Plots. These Dot Plots display examples in an ascending number order as displayed on the protocol given in Example 1. The Dot Plots correspond to the analysis of the whole blood from an allergic patient (obtained on a BD LSRII flow cytometer equipped with the DIVA Software).

FIG. 3. Calculation of the ex vivo frequency of allergen-specific CD4+ T cells.

FIG. 4. Representative example of ex vivo DR4/Phlp1 and DR4/Phlp5 tetramer staining in grass pollen allergic individuals. Freshly isolated peripheral blood mononuclear cells (PBMC) from DR4 allergic subjects were stained with PE-labeled MHC-class II tetramers and then enriched using anti-PE microbeads. Plots are gated on CD4+ CD14-CD19-Via-Probe-cells. Irrelevant-peptide tetramer staining was used as a negative control.

FIG. 5. Ex vivo frequencies of DR4-Timothy grass allergen-specific CD4+ T cells in grass pollen-allergic individuals (n=7) during the grass pollen season. Frequency was calculated by dividing the number of CD4+ tetramer+ cells after enrichment by the input number of CD4+ cells.

FIG. 6. Ex vivo phenotypic analysis between timothy grass allergen-specific CD4+ T cells. Freshly isolated PBMCs from allergic subjects (n=7) were stained with PE-labeled MHC-class II tetramers and then enriched using anti-PE microbeads. Cells were then stained with a combination of antibodies directed against the indicated surface markers. Results are expressed as mean percentages of CD4+ timothy grass tetramer-positive cells expressing the various surface markers. Error bars represent SEM.

FIG. 7. Representative example of intracellular staining between a) Phl p 1 120-139 and b) Phl p 5b 197-216-specific CD4+ T cells from same allergic individual. PBMCs from DR4-allergic subjects were stimulated with an immunodominant epitope from either Phl p 1 or Phl p 5 allergens for 2 weeks and then stained with the corresponding DR4-peptide tetramers. Cells were subsequently stimulated with PMA/IONO for 6 hrs and Brefeldin A was added after the first hour of stimulation. For cytokine analysis cells were stained with anti-IL4, anti-IL10 and anti-IFNγAb after mild permeabilization. Data are representative of 5 independent experiments.

FIG. 8. Identification of DR2a-restricted Aln g 1 specific CD4+ T cell epitope. A) Example of pooled mapping results of DR2a-restricted Aln g 1 epitopes. PBMC from a DR15/DRB5 alder pollen allergic subject were stimulated with 4 pools of Aln g 1 peptides (five 20 mer peptides per pool) for 2 weeks and then stained with the corresponding DR2a/Aln g 1 pooled peptide tetramers. B) Example of fine mapping results of DR2a-restricted Aln g 1 epitopes. Cells stimulated with peptide pools that gave positive staining were stained with DR2a individual peptide tetramers. Data are representative of three independent experiments. Subsequent experiments identified Aln g 1₁₄₂₋₁₅₄ as the minimal epitope, identical to the previously-identified DR2b restricted epitope.

FIG. 9. DR2b- and DR2a-restricted Aln g 1-specific CD4+ cells analysis. A) Ex vivo tetramer staining of DR2b- and DR2a-restricted Aln g 1 specific CD4+ cells. PE-labeled DR2b/Aln g 1₁₄₂₋₁₅₄ and PE-labeled DR2a/Aln g 1₁₄₂₋₁₅₄ tetramers were being used in these experiments. Freshly isolated PBMC from alder allergic subjects were first incubated with PE-labeled tetramers, and subsequently with anti-PE magnetic beads. PE-tetramer labeled cells were enriched with magnetic column. The cells were subsequently flushed out, and analyzed by flow cytometry. B) Ex vivo frequency of DR2b- and DR2a-restricted Aln g 1 specific CD4+ cells.

FIG. 10. Phenotypes of DR2b- and DR2a-restricted Aln g 1-specific CD4+ T cells. The anti-PE magnetic bead enrichment protocol was used to examine the surface phenotype of DR2b- and DR2a-restricted Aln g 1 CD4+ T cells in PBMC ex vivo. The surface markers being used were CD45RO, CD27, CRTh2 and CCR4. Results are expressed as mean percentages of CD4+ Aln g 1 tetramer-positive expressing the various surface markers. Error bars represent SEM.

FIG. 11. IL-5 production of DR2b and DR2a-restricted Aln g 1 specific CD4+ T cells. Freshly-isolated PBMCs from a DR15/DRB5-alder pollen-allergic subject were stained with specific PE-labelled MHC-class II tetramers and then enriched using anti-PE microbeads. IL-5 secreting cells were then evaluated with the Miltenyi IL-5 cytokine capture assays. Results are expressed as percentages of MHC-class II Aln g 1₁₄₂₋₁₅₄ tetramer+CD4+ T cells producing the respective cytokine. Data are representative of three independent experiments.

FIG. 12. Direct ex vivo analysis of Phl p reactive pathogenic T cells. The TGEM approach was used to identify Phl p specific T cell epitopes for different HLA, including HLA-DR0101, HLA-DR0301, HLA-DR0701 and HLA-DR1101. Multiple Phl p T cell epitopes restricted by the 4 alleles were identified. However, T cells that recognize these different epitopes have different phenotypic and functional properties. Thus, it was characterized whether the epitope identified is a Th2 epitope as defined by the surface expression of CRTH2. Using ex vivo tetramer staining of PBMC from Timothy grass allergic subjects, it was demonstrated that KGSNPNYLALLVKYVNGDGD (SEQ ID NO:22) and KLIEDINVGFKAAVAAAASV (SEQ ID NO:23) are DR0101 restricted Th2 epitopes; GDGDVVAVDIKEKGKDKWIE (SEQ ID NO:24) is a DR0301 restricted Th2 epitope; PEAKYDAYVATLSEALRIIA (SEQ ID NO:25) and ATPEAKFDSFVASLTEALRV (SEQ ID NO:26) are DR0701 restricted Th2 epitopes; and FAEGLSGEPKGAAESSSKAA (SEQ ID NO:27) is a DR1101 restricted Th2 epitope.

FIG. 13. Frequencies of Ara h 1 epitope-reactive T cells. A) Frequencies of Ara h 1₃₂₁₋₃₄₀-specific T cells in a DR1101 allergic subject and a DR1101 non-atopic subject. The frequencies of Ara h 1-specific T cells per million CD4+ T cells are as indicated. B) Frequencies of Ara h 1 epitope-reactive T cells in 11 peanut allergic subjects, 6 non-atopic subjects and 5 peanut non-allergic atopic subjects. Each data point represents the frequency of T cells specific for a single epitope in Ara h 1. A student t test was used in the statistical analysis. *P<0.05.

### DETAILED DESCRIPTION OF THE PREFERRED EMBODIMENTS

Provided herein are methods for the determination of antigen-specific CD4+ T cell phenotype and/or frequency based upon antigen-specific CD4+ T cells. This determination is useful for, inter alia, detecting or monitoring immune function, directing immunotherapy to the use of those epitopes or antigen fragments that elicit an allergic reaction (e.g., as measured by detection of a Th2 response) and/or promote immune deviation, monitoring an immune response to a particular antigen, etc., and in some embodiments is based upon a subject's MHC class II genotype.

The disclosures of all United States patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” or “/” refers to, and encompasses any and all possible combinations of one or more of the associated listed items, as well as the lack of combinations when interpreted in the alternative (“or”).

1. Peptides that Elicit a Th2 Immune Response

In some embodiments, “CD4+ Th2 eliciting polypeptides” are provided, the presence of which is associated with an increase in the number or frequency of the Th2 phenotype of CD4+ T cells in a subject and/or blood sample. In some embodiments, these polypeptides are small fragments of an antigen, e.g., 5, 6, 7, 8, 9, 10, 11, 12, 13, 14, 15, 16, 17, 18, 19, 20, 21, or 22 amino acids long in some embodiments, which fragment elicits a Th2 immune response in a subject (e.g., a subject with an allergy to the antigen), or homologues thereof. In some embodiments, polypeptides are 9 or more amino acids long (e.g., CD4+ T cell epitopes).

An “immune response” elicited, detected and/or monitored can be a protective immune response, a cellular immune response, a humoral immune response, a Th1 immune response, a Th2 immune response, or any combination thereof. Detection and/or monitoring of the immune response may be useful, e.g., in monitoring immune function and/or response to an antigen, e.g., for autoimmune disease, cancer, diabetes or multiple sclerosis, or to test the effectiveness of a vaccine or vaccination.

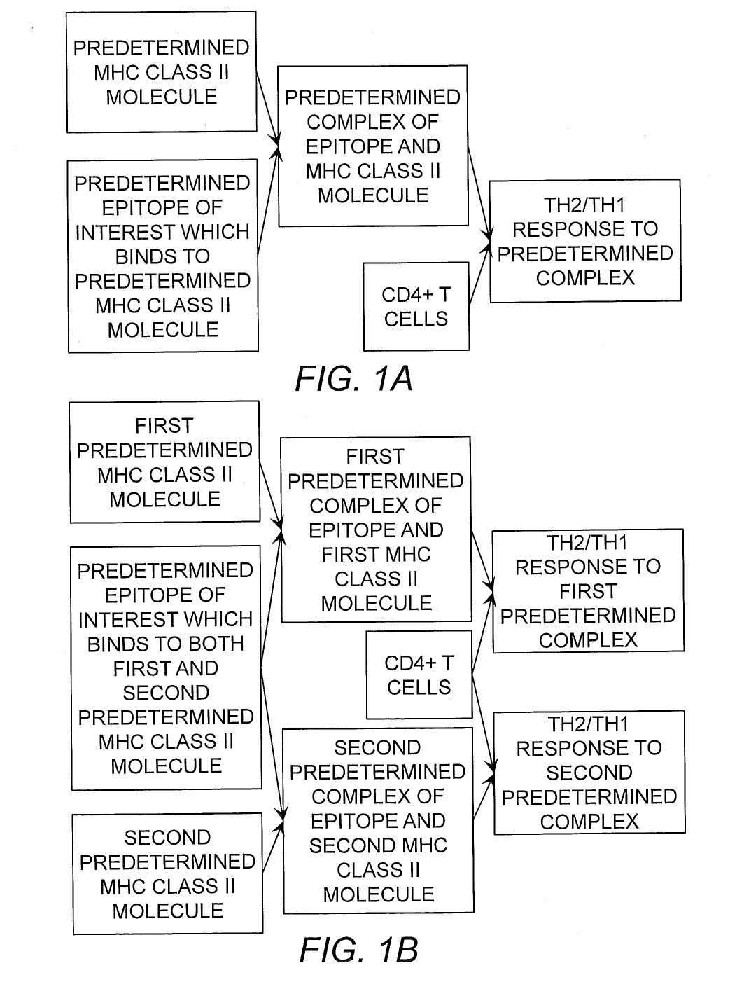

In some embodiments of the present invention, a predetermined complex of a predetermined MHC class II molecule and a predetermined antigen is used to probe a CD4+ T cell response thereto (see FIG. 1A). A “predetermined” MHC class II molecule as used herein means that the amino acid sequence (and HLA genotype) of the MHC class II molecule is known. Similarly, a “predetermined” antigen, antigenic fragment or epitope means that the amino acid sequence thereof is known. The “predetermined complex” is the predetermined antigen, antigenic fragment or epitope bound to the predetermined MHC class II molecule.

An “antigen” as used herein is a molecule or molecule fragment that is able to bind specifically to a major histocompatibility complex (MHC) molecule for presentation to the immune system cells, which complex of molecule or molecule fragment and MHC molecule in turns binds to immune system cell receptors (e.g., T cell receptors). An “immunogen” is a particular type of antigen that is able to provoke a humoral and/or cell mediated immune response if injected on its own. Antigens are usually proteins or polysaccharides, including parts (coats, capsules, cell walls, flagella, fimbrae, and toxins) of bacteria, viruses, and other microorganisms. Lipids and nucleic acids are normally only antigenic when combined with proteins and polysaccharides. Non-microbial exogenous (non-self) antigens can include pollen, egg white or other food antigens, and proteins from transplanted tissues and organs or on the surface of transfused blood cells. A “vaccine” is an example of immunogenic antigens intentionally administered to induce acquired immunity in the recipient.

An “immunogenic fragment” is a fragment of an immunogen that can stimulate a humoral and/or cellular immune responses in the subject. An immunogenic fragment can comprise, consist essentially of and/or consist of one, two, three, four or more epitopes. An immunogenic fragment can be any fragment of contiguous amino acids of an antigen and can be for example, 5, 10, 15, 20, 25, 30, 35, 40, 45, 50, 75, 80, 85, 90, 95, 100, 125, 150, 175, 200, 250, 300, 350, 400, 450, 500, 550, 600 or more amino acids in length, including any number between or beyond those numbers recited herein (e.g., 9, 23, 47 or 468 amino acids). Identification of any such immunogenic fragments is routine in the art.

The term “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., 75%, 80%, 85%, 90%, 92%, 95%, 98%, 99% identical) to the reference polypeptide or amino acid sequence. Such a polypeptide fragment may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, 250, 300, 350, 400, 450, 500 or more consecutive amino acids of a polypeptide or amino acid sequence.

An “epitope” is that portion or portions of an antigen which has a minimum molecular structure able to be recognized by the immune system, particularly by T cells, antibodies, and/or B cells. An epitope may be three-dimensional, such that the antigenic property is determined by its overall three-dimensional structure (tertiary structure), or linear, where the antigenic property is determined by a specific amino acid sequence (primary structure). An epitope is not limited to a polypeptide having the exact sequence of the portion of the parent protein from which it is derived, but encompasses sequences identical to the native sequence, as well as modifications to the native sequence, such as deletions, additions and substitutions (generally, but not always, conservative in nature).

“T cell epitopes” can bind to major histocompatibility complex (MHC) molecules and be presented on the surface of an antigen-presenting cell. Many T cell epitopes are known and publicly available from online databases such as MHCBN, SYFPEITHI, IEDB, ANTIJEN (Jenner Institued, United Kingdom), and IMGT/3Dstructure-DB (Montpellier, France).

Epitope homologues are also contemplated. An amino acid sequence or protein is defined as a “homologue” of an antigen, antigenic fragment or epitope if it has significant homology or identity and/or significantly similar three-dimensional structure to preserve the biological function thereof. Significant homology or identity means at least 50%, 55%, 60%, 65%, 70%, 75%, 80%, 85%, 90%, 95%, 98% and/or 100% homology or identity with another amino acid sequence. Such homologues can also be identified by having significant identity at the nucleotide sequence level (i.e., at least 50%, 55%, 60%, 65%, 70%, 75%, 80%, 85%, 90%, 95%, 98% and/or 100% or identity with another nucleotide sequence). Three-dimensional structure of a protein can be determined through computer data analysis as known in the art.

Epitopes can be “mapped,” the process of identifying and characterizing the minimum molecular structures that are able to be recognized by the immune system, according to methods known in the art, e.g., protein microarrays, ELISPOT or ELISA techniques, etc. In some embodiments, T cell epitopes which bind to MHC class II molecules are mapped with the use of Tetramer Guided Epitope Mapping (TGEM). See U.S. Pat. No. 7,094,555 to Kwok et al., which is incorporated by reference herein in its entirety. Epitopes may also be predicted based upon computer modeling, e.g., the TEPITOPE program. See, e.g., Kwok et al., Trends in Immunology, 2001, 22(11): 583-588.

In some embodiments, CD4+ T cells activated by a predetermined complex of a predetermined MHC class II molecule and a predetermined antigen, antigenic fragment or epitope may be characterized and/or sorted based upon markers of a Th1 and/or Th2 response. In some embodiments, markers are detected using a suitable immunological technique, e.g., flow cytometry for membrane-bound markers, immunohistochemistry for intracellular markers, and enzyme-linked immunoassay for markers secreted into the medium. The expression of protein markers can also be detected at the mRNA level by, e.g., reverse transcriptase-PCR using marker-specific primers. See, e.g., U.S. Pat. No. 5,843,780.

T cells can be provided in a biological sample from a subject. Suitable samples can include, for example, blood, lymph, lymph nodes, spleen, liver, kidney, pancreas, tonsil, thymus, joints, synovia, and other tissues in which T cells may be found. T cells may be isolated as peripheral blood mononuclear cells (PBMC). PBMC can be partially purified, for example, by centrifugation (e.g., from a buffy coat), by density gradient centrifugation (e.g., through a Ficoll-Hypaque), by panning, affinity separation, cell sorting (e.g., using antibodies specific for one or more cell surface markers), and other techniques that provide enrichment of PBMC and/or T cells.

In some embodiments, and for ease of use, the biological sample provided for the testing disclosed herein is whole blood without prior purification and/or enrichment of PBMC and/or T cells. In some embodiments, only a small sample is required for testing as described herein, e.g., between 0.1, 0.5, 1 or 2, and 3, 5, 8 or 10 milliliters of a whole blood sample.

Some embodiments make use of MHC class II tetramer staining in order to detect the CD4+ T cell phenotype and/or frequency based upon a predetermined MHC class II molecule-antigen complex. Major histocompatibility complex (MHC) class II tetramers allow the direct visualization of antigen specific CD4+ T cells by flow cytometry. See U.S. Pat. No. 7,094,555 to Kwok et al., which is incorporated by reference herein. This method relies on the highly specific interaction between the peptide-loaded MHC class II molecule and its corresponding T-cell receptor. While the affinity of a single MHC/peptide molecule is low, cross-linking MHC/peptide complexes with streptavidin increases the affinity of the interaction, enabling their use as staining reagents.

In some embodiments, detecting whether activated CD4+ T cells produce a Th1 and/or Th2 response may be performed by detecting the cytokines produced. Production of “Th2” cytokines is associated with allergic disease including asthma. Cytokines associated with Th2 include interleukin-4, interleukin-5, interleukin-6, interleukin-10, and interleukin-13. In contrast, cytokines associated with “Th1” are associated with a normal, non-allergic response to an antigen. Cytokines associated with Th1 include interferon-γ and tumor necrosis factor-beta.

In some embodiments, the assay makes use of flow cytometry (e.g., FACS) for analyzing CD4+ T cells. “CD4+ T cells,” also known as “helper T cells” or “Th,” are a type of white blood cell and express the CD4 protein on their surface. Activated CD4+ T cells differentiate into two major subtypes, “Type 1” (“Th1”) and “Type 2” (“Th2”). CD4+ T cells in some embodiments are analyzed with a gating tool configured to detect and gate based upon predetermined markers and/or molecule detection.

In some embodiments, the combination of CD27, CD45RO and/or CRTH2 is used as Th2 markers. The analysis of markers such as CRTH2, CD45RO and/or CD27 within allergen-specific CD4+ T cells allows the determination of the allergen-specific T cells subset (Th1 or Th2). This, in turn, may be used to predict the effectiveness of allergen-specific immunotherapy in a patient during desensitization based upon the patient's HLA genotype. For example, after excluding monocytes, macrophages, dendritic cells and B cells (CD 14 and CD19 positive cells), CD4 memory T lymphocytes may be analyzed using CD4 and CD45RO expression. Allergen-specific CD4+ Th2 cells can be identified as CD4^{pos} MHC-class II tetramer^{pos} CRTH2^{pos} CD27^{neg}, whereas non-Th2 allergen-specific CD4+ T cells can be identified as CD4^{pos} MHC-class II tetramer^{pos} CRTH2^{neg} CD27^{pos}.

CD 14 is a surface protein preferentially expressed on monocytes/macrophages. It binds lipopolysaccharide binding protein and recently has been shown to bind apoptotic cells.

CD19 is a cell surface molecule expressed only by B lymphocytes and follicular dendritic cells of the hematopoietic system.

CD4 is a glycoprotein expressed on the surface of T helper cells, regulatory T cells, monocytes, macrophages, and dendritic cells. T cells expressing CD4 are also known as CD4⁺ T cells. CD4 is a co-receptor that assists the T cell receptor (TCR) to activate its T cell following an interaction with an antigen presenting cell. Using its portion that resides inside the T cell, CD4 amplifies the signal generated by the TCR by recruiting an enzyme, known as the tyrosine kinase lck, which is essential for activating many molecules involved in the signaling cascade of an activated T cell. CD4 also interacts directly with MHC class II molecules on the surface of the antigen presenting cell using its extracellular domain.

CRTH2 (Chemoattractant receptor-homologous molecule expressed on Th2 lymphocytes) is a cognate receptor for prostaglandin (PG) D₂. The high expression levels of CRTH2 in Th2 lymphocytes, basophils and eosinophils imply a major role of CRTH2 in allergic diseases.

CD45RO is expressed on CD4⁺ and CD8⁺ T memory cells as well as on CD4⁺ effector T cells. CD45RO is also expressed on monocytes, macrophages, and granulocytes.

CD27 is a member of the TNF-receptor superfamily. This receptor is required for generation and long-term maintenance of T cell immunity. It binds to ligand CD70, and plays a key role in regulating B-cell activation and immunoglobulin synthesis.

An “allergy” or “allergic disorder” is a disorder in which the immune system is hypersensitive to normally harmless environmental substances. These environmental substances that cause allergies are called “allergens.” Common allergic diseases include seasonal rhinoconjuctivitis (e.g., allergies to grasses and pollen such as ragweed, timothy grass), allergies to pet dander such as cat dander or dog dander, food allergies such as peanut, dairy and wheat allergies, venum anaphylaxis, and asthma. Production of the “IgE” form of antibody is associated with allergic reaction as well as anaphylactic shock.

Though not wishing to be bound by theory, an allergic reaction begins when an MHC class II molecule of an antigen presenting cell binds to and presents an allergen or portion thereof to CD4+ T cells of the immune system, which in turn elicit an over-reactive immune response to that allergen. The Th2 response is associated with an allergic reaction.

2. Immunotherapy

In some embodiments, peptides that elicit a Th2 response in a subject are useful in immunotherapy. The peptides may be provided in a composition comprising, consisting of or consisting essentially of the same. While not wishing to be bound by theory, use of more specific set of antigenic peptides is thought to be preferable for use in immunotherapy over a crude extract of an entire antigenic protein or a larger mixture of peptides, only some of which elicit a Th2 response in a subject.

Adverse reactions known to occur with the administration of crude extracts of antigens include local reactions of redness and swelling at the injection site, as well as systemic reactions, including allergy symptoms such as sneezing, runny nose, congestions, rash, etc. More serious systemic reactions may include anaphylaxis. While not wishing to be bound by theory, shorter and/or fewer peptides administered according to some embodiments may be beneficial by reducing these potential adverse reactions.

As used herein, the term “consists essentially of” or “consisting essentially of” means that the antigenic composition of this invention comprises no other material antigenic agent other than the indicated agent(s). The term “consists essentially of” does not exclude the presence of other components such as adjuvants, immunomodulators, and the like.

In some embodiments, compositions comprising the peptides have at least 20, 30, 40, 50, 60, 70, 80, 90, or 95% or more by weight of the total peptides in the composition being Th2 eliciting peptides, e.g., the Th2 eliciting peptides provided herein.

“Specific immunotherapy” or “allergen-specific immunotherapy” is the administration of gradually increasing doses of crude extracts of allergens, making subjects tolerant to them (see, e.g., Francis et al., Peptide-based vaccination: where do we stand? Curr Opin Allergy and Clin Immunology 2005, 5:537-543). Successful allergen-specific immunotherapy is associated with “immune deviation,” i.e., the switch from the allergen-specific Th2 response typical for allergic patients (e.g., CD4⁺ CRTH2⁺ CD27⁻) to a more Th1/Treg response characteristic for non-allergic individuals (e.g., CD4⁺ CRTHT2⁻ CD27⁺). In some embodiments, immune deviation is measured by an increase in the ratio of Th1/Th2 response, e.g., by 1, 5, or 10, to 20, 15 or 100-fold.

In some embodiments, immune deviation is measured by the change in the ratio of IgG to IgE antibodies specific for the allergen being administered. In some embodiments, immune deviation is measured by an increase in the IgG/IgE ratio, e.g., in which this ratio increases by 1, 5, or 10, to 20, 15 or 100-fold.

“Subjects” that may be treated and/or monitored by methods of the present invention include both human subjects for medical purposes and animal subjects for veterinary and laboratory purposes. Other suitable animal subjects are, in general, mammalian subjects such as primates, bovines, ovines, caprines, porcines, equines, felines, canines, lagomorphs, rodents (e.g., rats and mice), etc. Human subjects are the most preferred. Human subjects include fetal, neonatal, infant, juvenile, adult and geriatric subjects.

“Treating” refers to any type of treatment or prevention that imparts a benefit to a subject afflicted with or at risk of developing allergies or an allergic reaction to an antigen of interest, including improvement in the condition of the subject (e.g., in one or more symptoms), delay in the onset of symptoms or slowing the progression of symptoms, etc. As used herein, “treatment” is not necessarily meant to imply cure or complete abolition of symptoms, but refers to any type of treatment that imparts a benefit to a patient.

“Treatment effective amount”, “prevention effective amount”, “amount effective to treat”, “amount effective to prevent” or the like as used herein means an amount of the material or composition sufficient to produce a desirable effect upon a patient inflicted with an allergy. This includes improvement in the condition of the patient (e.g., in one or more symptoms), delay in the onset or progression of the disease or disorder, etc.

In some embodiments, peptides may be provided in a carrier (e.g., a pharmaceutically acceptable carrier). As used herein, by “pharmaceutically acceptable” is meant a material that may be administered to a subject without causing appreciable or undue undesirable or adverse biological effects. Side effects are “undue” when their risk outweighs the benefit provided by the composition. Such a pharmaceutical composition may be used, for example, to prepare compositions for immunization. Non-limiting examples of pharmaceutically acceptable carriers include any of the standard pharmaceutical carriers such as phosphate buffered saline solutions, water, emulsions such as oil/water emulsion, microemulsions and various types of wetting agents. Physiologically and pharmaceutically acceptable carriers may contain other compounds including, but not limited to, stabilizers, salts, buffers, adjuvants and/or preservatives (e.g., antibacterial, antifungal and antiviral agents) as are known in the art.

The pharmaceutically acceptable carrier can be sterile in some embodiments and/or formulated for delivery into and/or administration to a subject. The compositions of the present invention can also include other medicinal agents, pharmaceutical agents, carriers, diluents, immunostimulatory cytokines, etc. Actual methods of preparing such dosage forms are known, or will be apparent, to those skilled in this art.

The compositions of this invention can be administered to a cell of a subject (e.g., blood cells) or to a subject either in vivo or ex vivo. For administration to a cell of the subject in vivo, as well as for administration to the subject, the compositions of this invention can be administered orally, parenterally (e.g., intravenously), by intramuscular injection, by intraperitoneal injection, subcutaneous injection, transdermally, sublingually, extracorporeally, topically or the like. Also, the compositions of this invention can be applied to CD4+ T cells ex vivo (e.g., provided in a whole blood sample), which are isolated and/or grown from a subject's biological sample, according to methods well known in the art, or onto bulk peripheral blood mononuclear cells (PBMC) or various cell subfractions thereof from a subject.

In some embodiments, the CD4+ T cell phenotype and/or frequency based upon a particular MHC class II molecule-antigen complex is compared to that of another MHC class II molecule-antigen complex in which the antigen bound by the MHC class II molecule is the same or substantially the same while the MHC class II molecule is different (i.e., a different haplotype) (see FIG. 1B). In this instance, the difference in T cell response is matched to a specific MHC class II genotype.

“Genotyping” or genotype determination of subjects can be carried out in accordance with known techniques, e.g., as described in U.S. Pat. Nos. 6,027,896 and 5,508,167. Genotyping herein includes “phenotyping,” or determining the genotype by determining or detecting which protein or proteins are expressed (e.g., by using MHC class II isoform-specific antibodies).

An “MHC class II molecule” is a cell surface receptor/antigen presenter comprised of alpha and beta subunits. MHC class II molecule genotypes (HLA-DR) are known in the art. The alpha subunit is encoded by the HLA-DRA gene, and lacks functional variation. In contrast, the variable beta subunit encoded by HLA-DRB comes in various forms (beta-1, HLA-DRB1; beta-2, HLA-DRB2; beta-3, HLA-DRB3, beta-4, HLA-DRB4; and beta-5, HLA-DRB5). The HLA-DRB1 locus is ubiquitous and encodes a very large number of functionally variable gene products (HLA-DR1 to HLA-DR17).

HLA-DR is closely linked to HLA-DQ (i.e., linkage disequilibrium), thus in some embodiments the HLA-DR genotype may be predicted based upon the HLA-DQ genotype of a subject. Some common DR and DQ haplotypes are given below in Table 1:

28 (of 75) Most common DR-DQ haplotypes
in Caucasian Americans

| | | | | |
|---|---|---|---|---|
| DR | DR-DQ | DR | DQ | Freq |

| Serotype | haplotype | B1 | A1 | B1 | % |
|---|---|---|---|---|---|
| DR1 | DR1-DQ5 | 0101 | 0101 | 0501 | 9.1 |
| | | 0102 | 0101 | 0501 | 1.4 |
| | | 0103 | 0101 | 0501 | 0.5 |
| DR3 | DR3-DQ2 | 0301 | 0501 | 0201 | 13.1 |
| DR4 | DR4-DQ7 | 0401 | 0300 | 0301 | 5.4 |
| | | 0407 | 0300 | 0301 | 0.9 |
| | DR4-DQ8 | 0401 | 0300 | 0302 | 5.0 |
| | | 0402 | 0300 | 0302 | 1.0 |
| | | 0403 | 0300 | 0302 | 0.4 |
| | | 0404 | 0300 | 0302 | 3.9 |
| | | 0405 | 0300 | 0302 | 0.3 |
| DR7 | DR7-DQ2 | 0701 | 0201 | 0202 | 11.1 |
| | DR7-DQ9 | 0701 | 0201 | 0303 | 3.7 |
| DR8 | DR8-DQ4 | 0801 | 0401 | 0402 | 2.2 |
| | DR8-DQ7 | 0803 | 0601 | 0301 | 0.1 |
| DR9 | DR9-DQ9 | 0901 | 0300 | 0303 | 0.8 |
| DR10 | DR10-DQ5 | 1001 | 0104 | 0501 | 0.7 |
| DR11 | DR11-DQ7 | 1101 | 0505 | 0301 | 5.6 |
| | | 1103 | 0505 | 0301 | 0.3 |
| | | 1104 | 0505 | 0301 | 2.7 |
| DR12 | DR12-DQ7 | 1201 | 0505 | 0301 | 1.1 |
| DR13 | DR13-DQ6 | 1301 | 0103 | 0603 | 5.6 |
| | | 1302 | 0102 | 0604 | 3.4 |
| | | 1302 | 0102 | 0609 | 0.7 |
| | DR13-DQ7 | 1303 | 0505 | 0301 | 0.7 |
| DR14 | DR14-DQ5 | 1401 | 0104 | 0503 | 2.0 |
| DR15 | DR15-DQ6 | 1501 | 0102 | 0602 | 14.2 |
| | | 1502 | 0103 | 0602 | 0.7 |
| DR16 | DR16-DQ5 | 1601 | 0102 | 0502 | 1.0 |

The “genotype” may include one or both haplotypes. A “haplotype” refers to a genetic variant or combination of variants carried on at least one chromosome in an individual, and may include multiple contiguous polymorphic loci. A diploid genome carries a pair of haplotypes for any given genetic locus, with sequences inherited on the homologous chromosomes from two parents. These haplotypes may be identical or may represent two different genetic variants for the given locus.

3. Nucleic Acids

Isolated nucleic acids may be provided which encode an antigen, antigenic fragment or epitope, or a homologue of any of these, useful for production of the same. Such nucleic acids can be present in a vector, which can be present in a cell (e.g., a cell transformed by the introduction of a heterologous nucleic acid). The present invention further provides isolated nucleic acids, vectors and cells of this invention for use in the methods described herein. Thus, in some embodiments, a nucleic acid encoding an antigen, an antigenic fragment or epitope, or a homologue of any of these, can be introduced into a subject under conditions well known in the art, wherein the nucleic acid is expressed and the encoded product is produced to elicit an immune response in the subject, thereby treating and/or preventing an allergic disease. The nucleic acids, vectors and/or cells of this invention can be present in a composition comprising a pharmaceutically acceptable carrier.

The term “isolated” can refer to a nucleic acid, nucleotide sequence, polypeptide or fragment thereof that is substantially and/or sufficiently free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or fragment or nucleic acid in a form in which it can be used for the intended purpose (e.g., therapeutically and/or in a diagnostic or detection assay).

An “isolated cell” refers to a cell that is substantially and/or sufficiently separated from other components with which it is normally associated in its natural state. For example, an isolated cell can be a cell in culture medium (e.g., in vitro or ex vivo) and/or a cell in a pharmaceutically acceptable carrier of this invention. Thus, an isolated cell in some embodiments can be delivered to and/or introduced into a subject. In some embodiments, an isolated cell can be a cell that is removed from a subject and manipulated ex vivo and then returned to the subject.

By the terms “express,” “expressing” or “expression” with regard to a nucleic acid comprising a coding sequence, it is meant that the nucleic acid is transcribed, and optionally, translated. Typically, expression of a coding sequence of the invention will result in production of the polypeptide, fragment, or other product of the invention. The produced polypeptide, fragment or other product may function in intact cells without purification.

Some embodiments of present invention are explained in greater detail in the following non-limiting examples.

### Example 1

### MHC Class II Tetramer Assay

Whole blood MHC class II tetramer staining consists of an optimized combination of fluorescent monoclonal antibody reagents, MHC class II tetramer loaded with an allergen-specific major epitope, a lysing solution and a fixative solution.

It is intended for in vitro diagnostic use for the determination of allergen-specific CD4+ T cell phenotype and frequency based on accurate allergen-specific CD4+ T cells gating tool (CD14^{neg} CD19^{neg} CD4^{pos} CD45RO^{pos} MHC class II tetramer^{pos} CD27^{+/-} and CRTH2^{+/-}). It is performed on a whole blood specimen.

The assay is designed to identify and characterize allergen-specific CD4+ T cells by flow cytometry. For this purpose, a 5-color combination may be used. It is a mixture of 6-fluorescent monoclonal antibodies (CD14-PercP, CD19-PercP, CD4-FITC, CRTH2-AF647, CD45RO-PECy5 and CD27-PECy7) and PE-labeled MHC-class II tetramer reagent.

MHC-Class II Tetramer.

Major histocompatibility complex (MHC) class II tetramers allow the direct visualization of antigen specific CD4+ T cells by flow cytometry. This method relies on the highly specific interaction between peptide loaded MHC and the corresponding T-cell receptor. While the affinity of a single MHC/peptide molecule is low, cross-linking MHC/peptide complexes with streptavidin increases the avidity of the interaction, enabling their use as staining reagents.

Using a fixative solution with whole blood MHC class II-tetramer staining allows blood specimen preparation by fixing the cell suspension during erythrolysis. It may also be used for fixing the preparation before flow cytometry analysis.

Flow cytometric procedures preferably use monodispersed cell preparations with the removal of erythrocyte interference. A lysing solution for whole blood MHC class II-tetramer staining may be used for the lysis of red blood cells in the preparation of biological samples for flow cytometry analysis.

The cell population of interest is stained with MHC-class II tetramer loaded with allergen-specific major epitope of interest or with irrelevant epitope as negative control. Cells are subsequently stained with monoclonal antibodies. Erythrocytes are then lysed prior to flow cytometry analysis.

After excluding monocytes, macrophages, dendritic cells and B cells (CD14 and CD19 positive cells), CD4 memory T lymphocytes are analyzed using CD4 and CD45RO expression. Allergen-specific CD4+ Th2 cells are identified as CD4^{pos} MHC-class II tetramer CRTH2^{pos} CD27^{neg}, whereas non-Th2 allergen-specific CD4+ T cells are identified as CD4^{pos} MHC-class II tetramer^{pos} CRTH2^{neg} CD27^{pos}.

Peripheral blood samples are collected aseptically into a sterile evacuated blood collection tube with anticoagulant. Specimens should be stored at room temperature until processing (preferably less than 48 hours). The specimen should be homogenized by gentle agitation prior to pipetting.

For each test, one ml of whole blood is alloquated into 15 ml conical tubes. Ten μl of PE-labeled MHC-class II tetramer reagent is added. Each tube is gently vortexed and incubated for 2 hours at room temperature, protected from light. Added is 4 μl CD4-FITC, 4 μl CD14-PercP, 4 μl CD19-PerCP, 4 μl CD45RO-PECy5, 4 μl CRTH2-AF647, 4 μl CD27-PECy7 antibodies per each tube and incubated for 20 minutes at room temperature, protected from light. Then 100 μl of fixative solution is added into each tube and gently vortexed for approximately 5 seconds.

Ten ml of lysing solution (preferably at room temperature) is added to each tube and vortexed vigorously, and then incubated at room temperature for 10 minutes, protected from light. The tubes are then centrifuged for 5 minutes at 200 g, and aspirate the supernatant. The lysing steps are then repeated.

The cell pellet is resuspended with 1 ml of PBS, and the cells are transferred into a (12×75 mm) FACS tube. Data is then acquired on the flow cytometer. If not analyzed within one hour, processed samples are stored between 2-8° C., protected from light, and analyzed within 6 hours.

The flow cytometer is equipped to detect Forward Scatter, Side Scatter and the six following fluorochromes FITC, PE, PercP (or ECD or PE-Texas Red), PE-Cy5, PE-Cy7 and APC (or AF 647). A minimum of 4 fluorochromes is required: FITC, PE, PercP and APC.

Create Dot Plots as follows in order to characterize allergen-specific CD4+ T cells:

1. Create Dot Plot 1 as Forward Scatter vs Side Scatter.

2. Create Dot Plot 2 as CD4-FITC vs CD14/CD19-PercP,

3. Create Dot Plot 3 as CD4-FITC vs CD45RO-PECy5.

4. Create Dot Plot 4 as CRTH2-AF647 vs MHCII Tetramer-PE.

5. Create Dot Plot 5 as CD27-PECy7 vs MHCII Tetramer-PE.

Create regions as follows:
1. Dot Plot 1—Create an amorphous/polygonal Region A on Dot Plot 1 to include all lymphocytes and eliminate red blood cell debris, aggregates, monocyte and granulocytes.
2. Dot Plot 2—Create a rectilinear Region B on Dot Plot 2 to include all CD4^{pos} T lymphocytes while excluding CD4^{neg} and CD14/CD19^{pos} cells.
3. Dot Plot 3—Create a rectilinear Region C on Dot Plot 3 to include all CD4^{pos} CD45RO^{pos} events.
4. Dot Plot 4—Create Quadstat Region D to distinguish all clustered between CRTH2^{neg} MHCII-tetramer^{pos} CD4^{pos} CD45RO^{pos} CD14/CD19^{neg} and CRTH2^{pos} MHCII-tetramer^{pos} CD4^{pos} CD45RO^{pos} CD14/CD19^{neg}.
5. Dot Plot 5—Create Quadstat Region E to distinguish all clustered between CD27^{pos} MHCII-tetramer^{pos} CD4^{pos} CD45RO^{pos} CD14/CD19^{neg} and CD27^{pos} MHCII-tetramer^{pos} CD4^{pos} CD45RO^{pos} CD14/CD19^{neg}.

Create gates as follows:
1, Dot Plot 1—Ungated to display all events.
2. Dot Plot 2—Assign “A” to Dot Plot 2 to display all lymphocytes while eliminating red blood cell debris, aggregates, monocyte and granulocytes.
3. Dot Plot 3—Assign “A” and “B” (AB) to Dot Plot 3 to display all CD4^{pos} T lymphocytes while excluding CD4^{neg} and CD14/CD19^{pos} cells.
4. Dot Plot 4—Assign “A”, “B” and “C” (ABC) to Dot Plot 4 to display all CD4^{pos} CD45RO^{pos} CD14/CD19″ lymphocytes, and show the MHCII-tetramer^{pos}CRTH2^{pos} events.
5. Dot Plot 5—Assign “A”, “B” and “C” (ABC) to Dot Plot 5 to display all CD4^{pos} CD45RO^{pos} CD14/CD19^{neg} lymphocytes, and show the MHCII-tetramer^{pos}CD27^{neg} events.

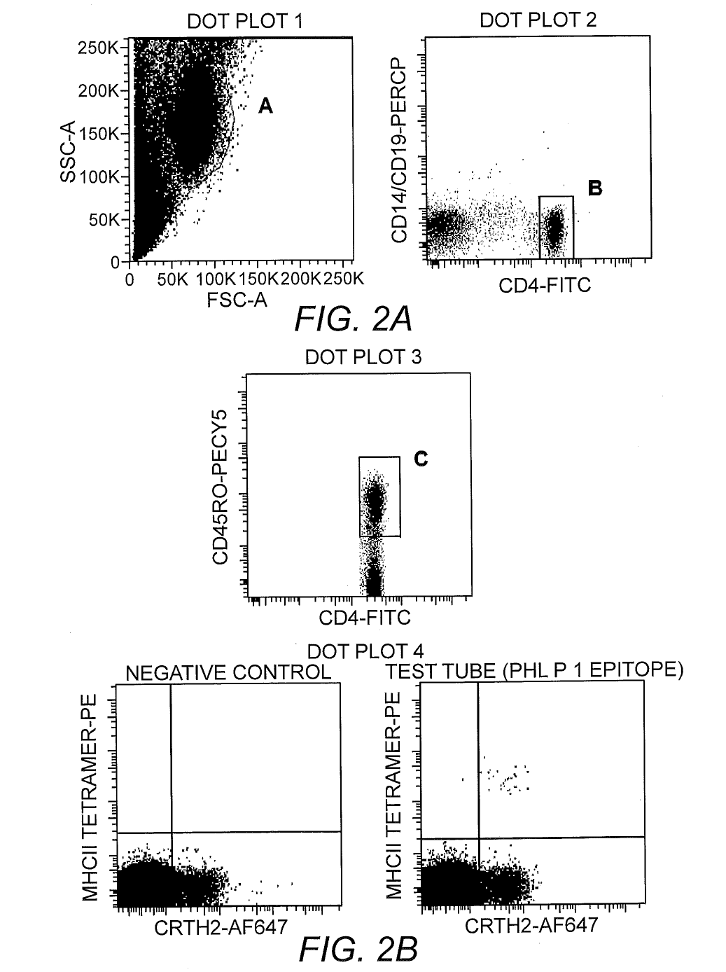

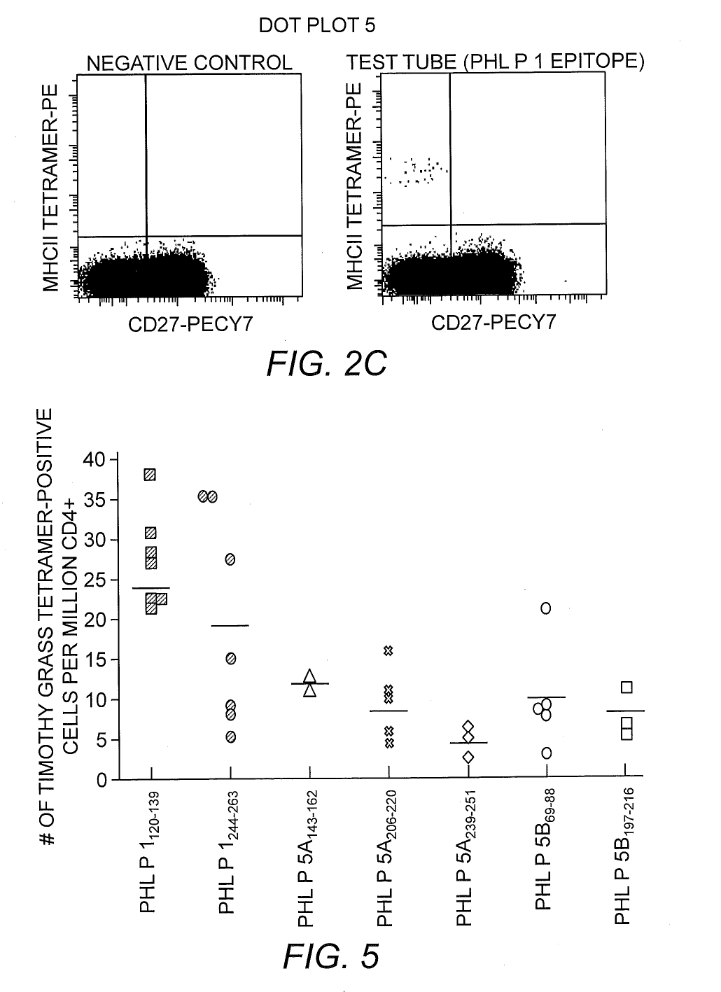

FIG. 2A-2C gives exemplary Dot Plots in an ascending number order as displayed on the protocol. To calculate allergen-specific CD4+ T cell frequencies, determine the total number of CD4^{pos} T cells in Dot Plot 3 (in gate AB); then determine the total of MHC II-tetramer^{pos} in Dot Plot 4 or 5. Allergen-specific CD4+ T cell frequency=(# of MHC-tetramer+ T cells)/(# of CD4+ T cells)

### Example 2

### Heterogeneity of Phl p 1 and Phl p 5 Reactive T Cells in Subjects with Timothy Grass Allergy

Grass pollen is a major cause of seasonal allergies in many parts of the world. In this study, we identified major antigenic epitopes of timothy grass allergen Phl p 1, Phl p 5a and Phl p 5b in HLA-DRB1*0401 individuals. Using HLA-DR*0401 tetramers loaded with these peptides, it was observed that timothy grass-allergic individuals consistently exhibited detectable numbers of CD4+ timothy grass tetramer-positive cells ex vivo in peripheral blood. Further analysis of CD4+ timothy grass tetramer-positive cells demonstrated functional and phenotypic heterogeneity between those different timothy grass epitope-specific CD4+ T cells from the same allergic individual.

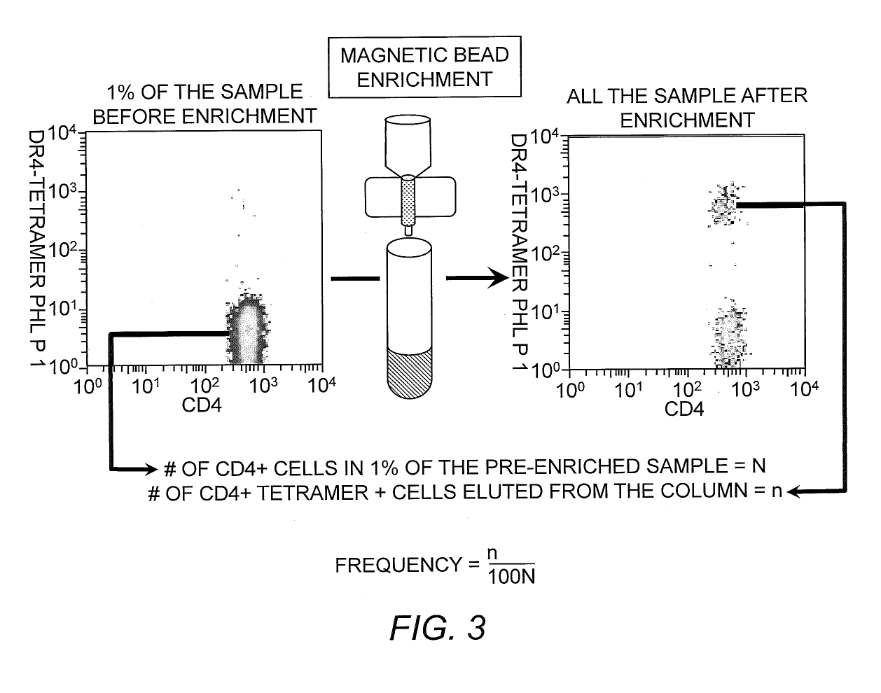

Grass pollen allergic subjects were recruited based on their clinical symptoms, a positive prick test and a positive IgE reactivity using the ImmunoCap test with grass pollen extracts (testscore≧3). Tetramer Guided Epitope Mapping was used to identify the antigenic peptides for the major grass pollen allergen proteins Phl p 1, Phl p 5a and Phl p 5b. For ex vivo detection of timothy grass-specific CD4+ T cells, freshly isolated peripheral blood mononuclear cells (PBMC) were stained with PE-labeled MHC-class II tetramers specific for these epitopes and were enriched using anti-PE microbeads. Ex vivo frequency was calculated by dividing the number of CD4+ tetramer+ cells after enrichment by the input number of CD4+ cells (FIG. 3).

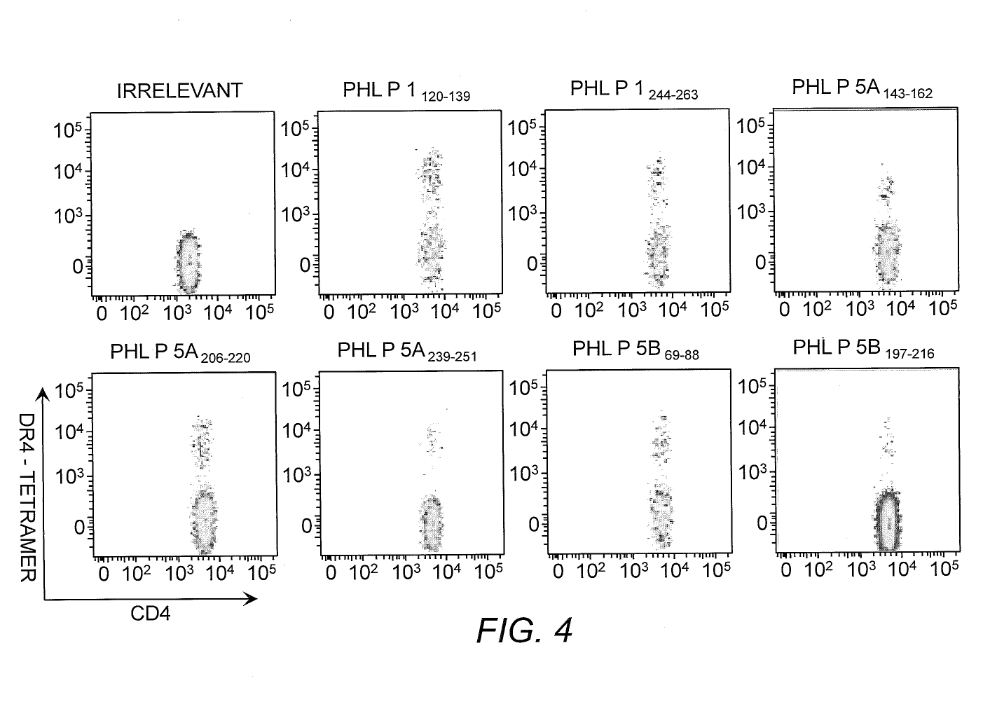

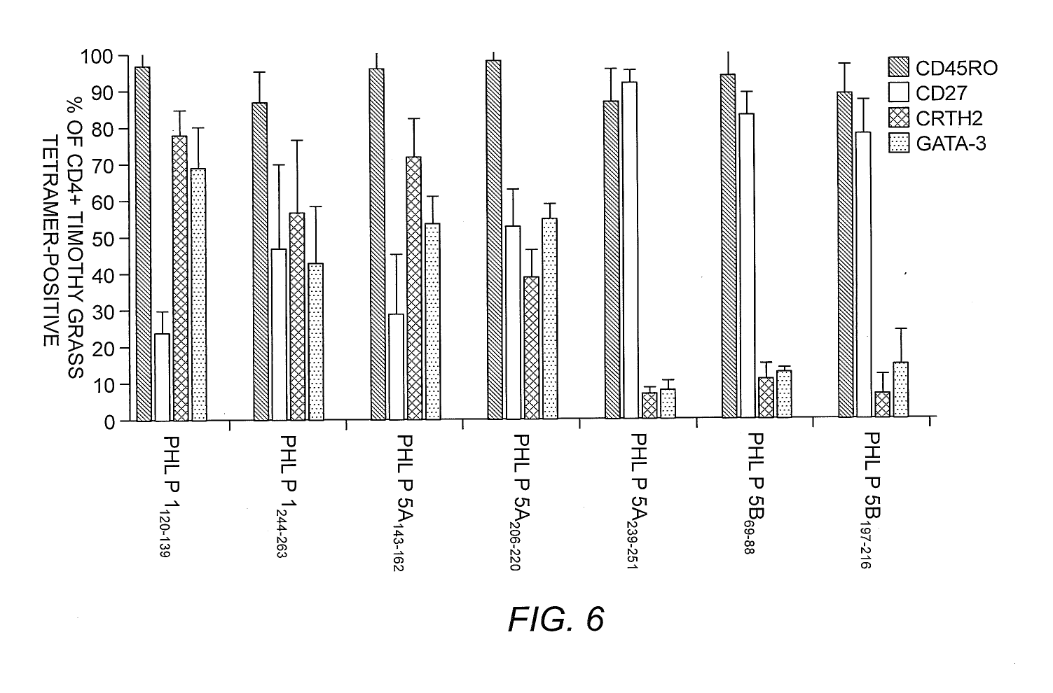

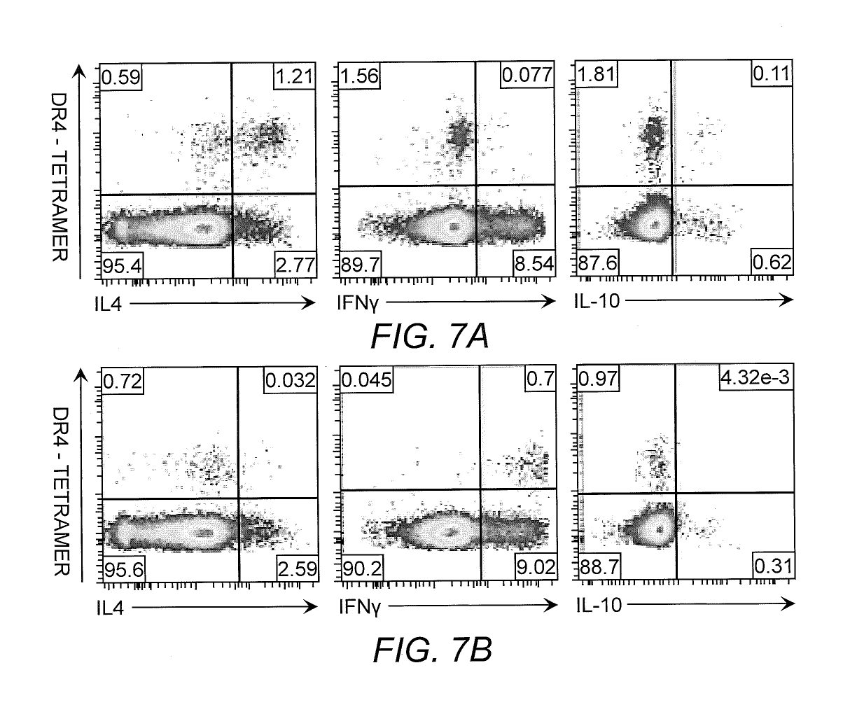

Surface marker phenotype of these Phl p 1 and Phl p 5 reactive CD4+ T cells were directly analysed by flow cytometry. Cytokine profiles were determined using intracellular staining after 2 weeks expansion with corresponding epitope. CD4+ T cell responses to Timothy grass allergens are directed to a broad range of epitopes. Combining the use of MHC-class II tetramers that correspond to these epitopes with magnetic enrichment of tetramer labeled cells, CD4+ T cells reactive to these epitopes were detected ex vivo in all DR4 grass pollen-allergic individuals tested (FIG. 4). Depending on the epitope (FIG. 5), heterogeneity of CD4+ T cell subset was observed between allergen-specific T cells based on CD27, CRTH2 and GATA3 expression (FIG. 6). Cytokine profile data indicate that CD27-GATA3+ allergen-specific T cells exhibit a clear Th2 response to allergen and this subset is more prevalent in allergic subjects (FIG. 7a). In contrast, CD27+ GATA3-allergen-specific T cells are present at a much lower frequency in grass pollen allergic individuals but mainly produce IFNγ in response to allergen (FIG. 7b).

Direct ex vivo characterization of allergen-specific CD4+ T cells provides the most accurate representation of in vivo events against natural allergen exposure in grass pollen allergic individuals. Different epitopes from the same allergen can elicit T cells with distinct immune functions. These different T cells subsets should have different roles in exacerbating or down regulating the immune responses towards the allergen.

The specific amino acid sequences of the epitopes are given below in Table 2:

| Allergen | Amino acid sequence | Position | HLA-DRB1 genotype |
|---|---|---|---|
| Phl p 1 | EEPIAPYHFDLSGHAFGAMA | 97-116 | HLA-DRB1*0401 |
| | SEQ ID NO: 1 | | |
| Phl p 1 | TEAEDVIPEGWKADTSYESK | 221-240 | HLA-DRB1*0401 |
| | SEQ ID NO: 2 | | |
| Phl p 1 | WYGKPTGAGPKDNGGACGYK | 25-44 | HLA-DRB1*0402 |
| | SEQ ID NO: 3 | | |
| Phl p 5a | LDAAYKLAYKTAEGATPEAK | 105-124 | HLA-DRB1*1001 |
| | SEQ ID NO: 4 | | |
| Phl p 5b | DTYKCIPSLEAAVKQ | 182-196 | HLA-DRB1*0401 |
| | SEQ ID NO: 5 | | |

The specific amino acid sequences of the antigens are given below.

Phl p 1.0102 (SEQ ID NO: 6):
MASSSSVLLV VVLFAVFLGS AYGIPKVPPG PNITATYGDK
WLDAKSTWYG KPTGAGPKDN GGACGYKDVD KPPFSGMTGC
GNTPIFKSGR GCGSCFEIKC TKPEACSGEP
VVVHITDDNEEPIAPYHFDL SGHAFGAMAK KGDEQKLRSA
GELELQFRRV KCKYPEGTKV TFHVEKGSNPNYLALLVKYV
NGDGDVVAVD IKEKGKDKWI ELKESWGAIW RIDTPDKLTG
PFTVRYTTEGGTKTEAEDVI PEGWKADTSY ESK
Phl p 5 a or Phl p 5.0101 (SEQ ID NO: 7):
MAVHQYTVALFLAVALVAGPAASYAADLGYGPATPAAPAAGYTPATPAA
PAEAAPAGKATTEEQKLIEKINAGFKAALAAAAGVQPADKYRTFVATFG
AASNKAFAEGLSGEPKGAAESSSKAALTSKLDAAYKLAYKTAEGATPEA
KYDAYVATLSEALRIIAGTLEVHAVKPAAEEVKVIPAGELQVIEKVDAA
FKVAATAANAAPANDKFTVFEAAFNDAIKASTGGAYESYKFIPALEAAV
KQAYAATVATAPEVKYTVFETALKKAITAMSEAQKAAKPAAAATATATA
AVGAATGAATAATGGYKV
Phl p 5b or Phl p 5.0201 (SEQ ID NO: 8):
AAAAVPRRGPRGGPGRSYTADAGYAPATPAAAGAAAGKATTEEQKLIED
INVGFKAAVAAAASVPAADKFKTFEAAFTSSSKAAAAKAPGLVPKLDAA
YSVAYKAAVGATPEAKFDSFVASLTEALRVIAGALEVHAVKPVTEEPGM
AKIPAGELQIIDKIDAAFKVAATAAATAPADDKFTVFEAAFNKAIKEST
GGAYDTYKCIPSLEAAVKQAYAATVAAAPQVKYAVFEAALTKAITAMSE
VQKVSQPATGAATVAAGAATTAAGAASGAATVAAGGYKV

### Example 3

### CD4+ T Cells Specific for a Single Alder (Aln g 1) Epitope Exhibit Different Effector Functions Dependent on their HLA-DR Molecule Restrictions

The HLA-DR15 gene is in tight linkage disequilibrium with the DRB5 gene. Subjects with the HLA-DR15 haplotype express both DRA/DRB1*1501 (DR2b) and DRA/DRB5*0101 (DR2a) class II molecules. In this study, we identified a major antigenic epitope of alder pollen major allergen Aln g 1 presented by both DR2b and DR2a molecules. The hypothesis was then tested that allergen-specific CD4+ T cells restricted by these two different DR molecules may have different effector functions.

DR15 alder pollen allergic subjects (n=5) were recruited based on their clinical symptoms, a positive prick test and a positive IgE reactivity using the ImmunoCap test with grass pollen extracts (Table 3).

TABLE 3
HLA and allergic status of recruited subjects

| | | | Immunocap grass pollen |
|---|---|---|---|
| Donors | HLA-typing | Skin prick test score | specific IgE score |
| #1 | DR15/DR15 | 4+++ | 4 |
| #2 | DR15/DR11 | 4+ | 4 |
| #3 | DR15/DR9 | 4+ | 4 |
| #4 | DR15/DR13 | 4++ | 4 |
| #5 | DR15/DR11 | 4+ | 4 |

Tetramer Guided Epitope Mapping (TGEM) was used to identify the antigenic peptides for the major alder pollen allergen proteins Aln g 1 that can be presented by both DR2b and DR2a molecules. For ex vivo detection of Aln g 1-specific CD4⁺ T cells, freshly isolated peripheral blood mononuclear cells (PBMC) were stained with PE-labeled MHC-class II tetramers specific for this epitope and were enriched using anti-PE microbeads. Ex vivo frequency was calculated by dividing the number of CD4⁺ tetramer⁺ cells after enrichment by the input number of CD4⁺ cells. MHC-class II tetramers were further used to examine the cytokine profiles and ex vivo surface phenotypes of these Aln g 1-reactive T cells in alder pollen-allergic subjects with the DR15 haplotype.

TGEM determine that the DRB5 (DR2a) restricted Aln g 1 CD4+ T cell epitope was identical to the previously identified DR1501 (DR2b) restricted Aln g 1 T cell epitopes (FIG. 7). Thus the Aln g 1₁₄₂₋₁₅₄ epitope is being presented by both DR2b and DR2a molecules.

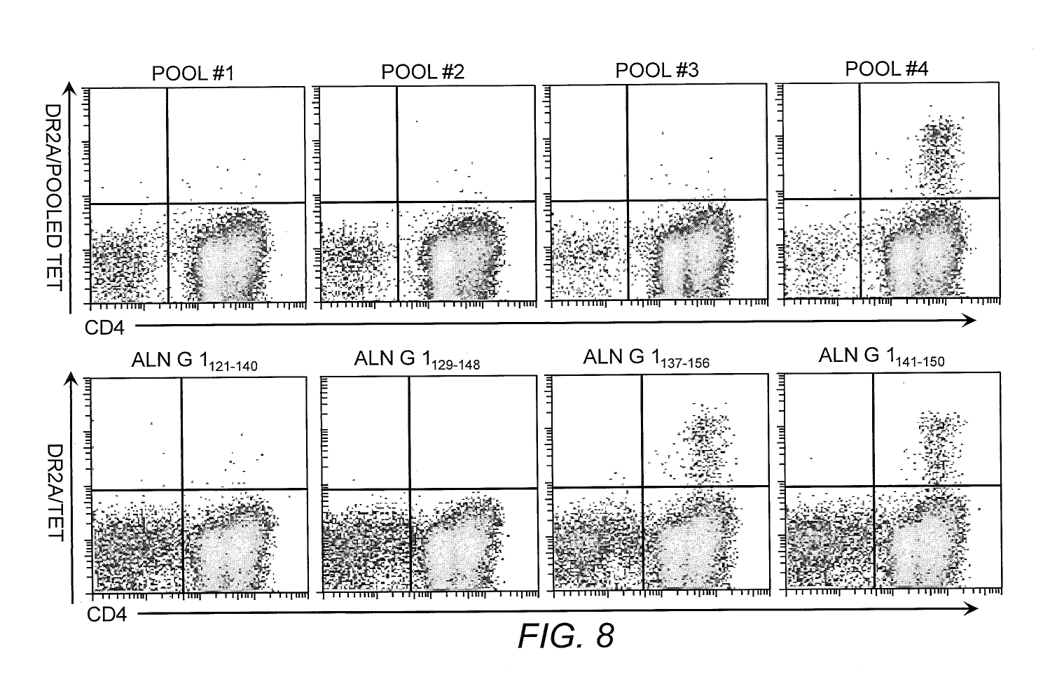

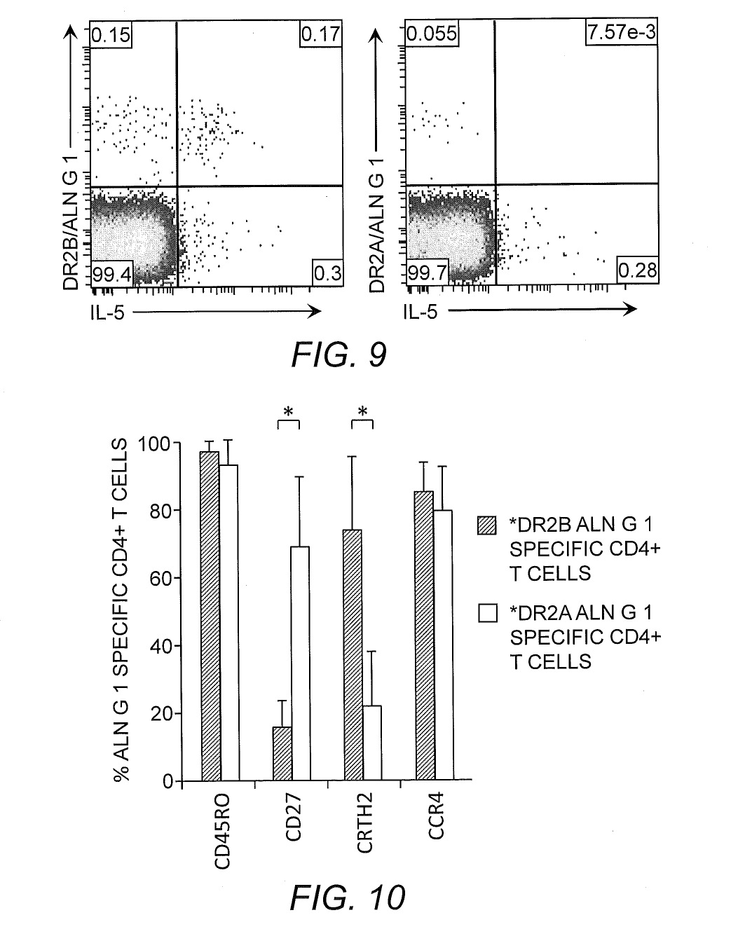

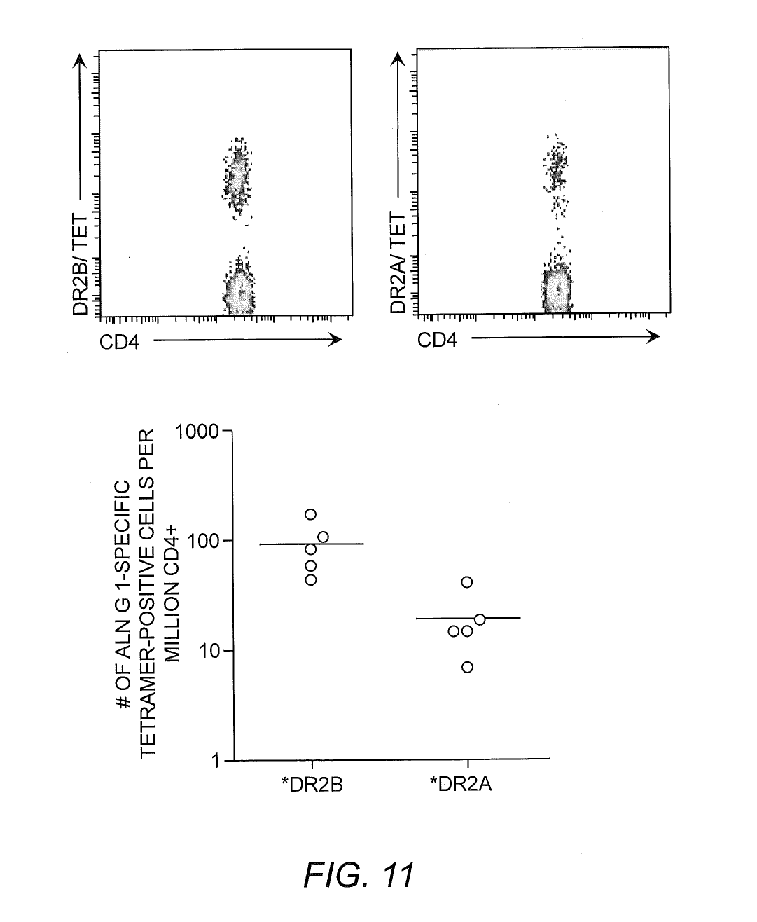

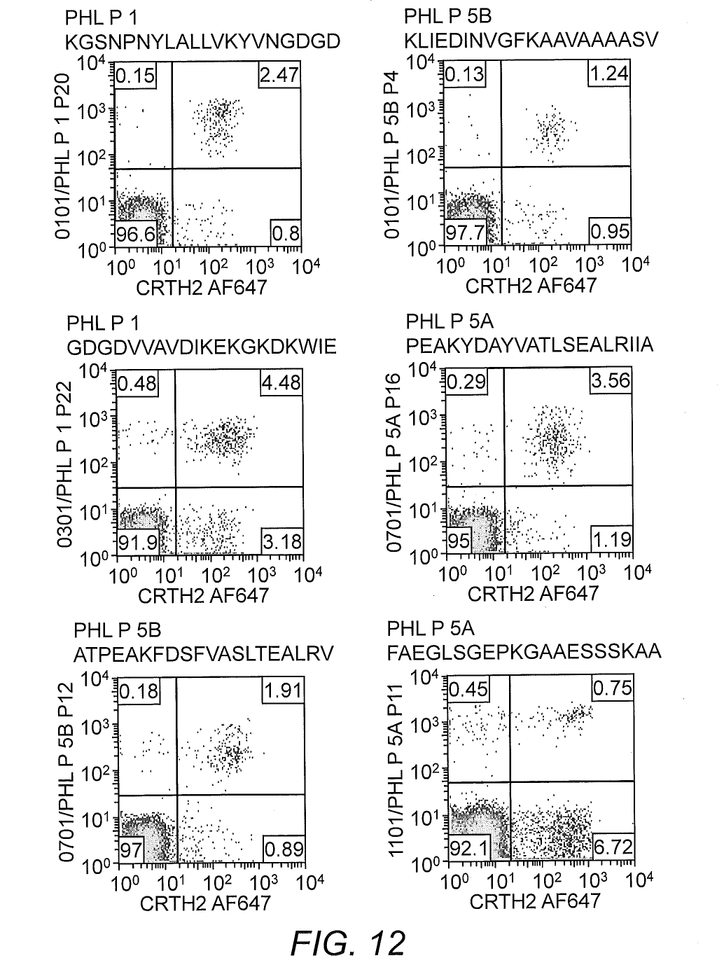

Both DR2b- and DR2a-restricted Aln g 1-reactive T cells specific for this epitope were detected ex vivo in DR15 alder pollen-allergic subjects. DR2b-restricted Aln g 1-reactive T cells were present at significantly higher frequencies compared to DR2a-restricted Aln g 1-reactive T cells (FIG. 10). DR2b-restricted T cells also expressed considerably higher levels of the Th2 marker CRTh2 than the DR2a-restricted T cells (FIG. 9). Using ex vivo functional assay, we also demonstrate that IL-5 secretion was only detected in DR2b/Aln g 1₁₄₂₋₁₅₄ CD4+ T cells, but not in DR2a/Aln g 1₁₄₂₋₁₅₄ CD4+ T cells (FIG. 8).

The Aln g 1₁₄₂₋₁₅₄ epitope is being presented by both DR2b and DR2a molecules. DR2b-restricted Aln g 1-specific T cells have a clear Th2 phenotypes as indicated by both surface markers and cytokine profile. In contrast, DR2a-restricted T cells have a “Th1” like phenotype under the current experimental condition. Both Th2 and “Th1” like Aln g 1 CD4⁺ specific T cells are present in DR15 alder pollen allergic subjects, with the Th2 cells being the dominant cell type.

TABLE 4
Aln g 1 peptides.

| Amino acid sequence | Position | HLA genotype |
|---|---|---|
| DRVNFKYSFSVIE | 76-88 | DR0701, DR0901 |
| SEQ ID NO: 9 | | |
| NFKYSFSVIEGGA | 79-91 | DR0701, DR0901 |
| SEQ ID NO: 10 | | |
| GSILKISNKFHTK | 112-124 | DR1101, DR0301 |
| SEQ ID NO: 11 | | |
| VGLLKAVESYLLA | 142-154 | DR1501, DR1502, |
| SEQ ID NO: 12 | | DR0701, DR0901, |
| | | DRB5 |
| LKAVESYLLAHSD | 145-157 | DR0901 |
| SEQ ID NO: 13 | | |
| VESYLLAHSDAYN | 148-160 | DR0901 |
| SEQ ID NO: 14 | | |

Aln g 1 antigen (SEQ ID NO: 15):
MGVFNYEAET PSVIPAARLF KAFILDGDKL LPKVAPEAVS
SVENIEGNGG PGTIKKITFPEGSPFKYVKE RVDEVDRVNF
KYSFSVIEGG AVGDALEKVC NEIKIVAAPD
GGSILKISNKFHTKGDHEIN AEQIKIEKEK AVGLLKAVES
YLLAHSDAYN

### Example 4

### Direct Ex Vivo Analysis of Feline Allergen-Specific CD4+ T Cells

To identify T cell epitopes within Fel d 1, the TGEM approach was applied to multiple allergic subjects recruited with informed consent from the Virginia Mason Medical Center Allergy Clinic and Benaroya Research Institute. Overlapping peptides corresponding to both chains of Fel d 1 were pooled and used to stimulate T cell cultures. Each peptide pool was loaded into purified class II molecules to generate tetramers. After 14 days, cultured cells were stained with corresponding pooled peptide loaded tetramers. Positive wells were stained again using tetramers loaded with single peptides. Applying this approach, we identified novel Fel d 1 epitopes for six HLA types (Table 5). Binding predictions and experiments using shorter peptides defined minimal epitopes. Responses to these Fel d 1 peptides were absent in non-allergic subjects.

The phenotype of allergen specific T cells was directly examined. The observations indicated that nearly all Fel d 1 specific T cells exhibit a memory phenotype. Fel d 1 specific T cells showed heterogeneous expression of CCR7, a marker thought to be up-regulated on central memory (TCM) and down-regulated on effector memory (TEM) cells. A previous report observed enriched CCR4 expression by allergen-specific T cells. Our results were more dramatic, in that almost all allergen-specific T cells were CCR4 positive (−25-30% of total CD4+ T cells were CCR4 positive). Expression of CCR4 is notable because CCR4 is a Th2 marker that has been associated with trafficking to non-lymphoid sites, including the skin and airway mucosa. Thus, high levels of CCR4 expression may lead to rapid recruitment into relevant sites for allergic immune responses.

In contrast with CCR4 expression, the prostaglandin D2 receptor CRTH2 (another Th2 marker) was expressed at variable frequencies (17% to 88%) amongst feline allergic subjects. While variable, these frequencies were always higher than total CD4+ T cells. Regardless of CRTH2 expression level, tetramer-based cytokine assays indicated high levels of IL-5 and low levels of γ-IFN. These cytokine results reinforced the surface phenotype results. Cytokine levels were robust, which is typical of effector T cells. The absence of CXCR3 and CCR6 expression indicates that these peripheral cells do not belong to Th1 or Th17 lineages. However, these lineages could be present within specific tissues or during stages of allergy that were not reflected in our samples.

TABLE 5
Fel d 1 T cell epitopes.

| HLA | | | |
|---|---|---|---|
| restriction | Epitope | AA sequence | |
| DRB1*0101 | Fel d 1 chain 1₂₅₋₄₄ | VAQYKALPVVLENARILKNC | SEQ ID NO: 16 |
| DRB1*0401 | Fel d 1 chain 2₂₂₋₃₁ | ELLLDLSLTK | SEQ ID NO: 17 |
| DRB1*1101 | Fel d 1 chain 1₅₈₋₆₇ | LSLLDKIYTS | SEQ ID NO: 18 |
| DRBP*1301 | Fel d 1 chain 1₃₁₋₄₃ | LPVVLENARILKN | SEQ ID NO: 19 |
| DRB1*1401 | Fel d 1 chain 1₅₅₋₆₇ | ENALSLLDKIYTS | SEQ ID NO: 20 |
| DRB5*0101 | Fel d 1 chain 1₁₉₋₃₁ | DEYVEQVAQYKAL | SEQ ID NO: 21 |

### Example 5

### Additional Phl p Epitopes

The TGEM approach was used to identify Phl p specific T cell epitopes for different HLA, including HLA-DR0101, HLA-DR0301, HLA-DR0701 and HLA-DR1101. Multiple Phl p T cell epitopes restricted by the 4 alleles were identified. However, T cells that recognize these different epitopes have different phenotypic and functional properties. Thus, it was characterized whether the epitope identified is a Th2 epitope as defined by the surface expression of CRTH2.

Using ex vivo tetramer staining of PBMC from Timothy grass allergic subjects, it was demonstrated that KGSNPNYLALLVKYVNGDGD (SEQ ID NO:22) and KLIEDINVGFKAAVAAAASV (SEQ ID NO:23) are DR0101 restricted Th2 epitopes; GDGDVVAVDIKEKGKDKWIE (SEQ ID NO:24) is a DR0301 restricted Th2 epitope; PEAKYDAYVATLSEALRIIA (SEQ ID NO:25) and ATPEAKFDSFVASLTEALRV (SEQ ID NO:26) are DR0701 restricted Th2 epitopes; and FAEGLSGEPKGAAESSSKAA (SEQ ID NO:27) is a DR1101 restricted Th2 epitope (Table 6).

TABLE 6
Phl p T cell epitopes.

| Antigen | Amino Acid Sequence | position | HLA restriction |
|---|---|---|---|
| Phl p 1 | KGSNPNYLALLVKYVNGDGD | 153-172 | HLA-DRB1*0101 SEQ ID NO: 22 |
| Ph1 p 5b | KLIEDINVGFKAAVAAAASV | 26-45 | HLA-DRB1*0101 SEQ ID NO: 23 |
| Ph1 p 1 | GDGDVVAVDIKEKGKDKWIE | 169-188 | HLA-DRB1*0301 SEQ ID NO: 24 |
| Ph1 p 5a | PEAKYDAYVATLSEALRIIA | 119-138 | HLA-DRB1*0701 SEQ ID NO: 25 |
| Ph1 p 5b | ATPEAKFDSFVASLTEALRV | 90-109 | HLA-DRB1*0701 SEQ ID NO: 26 |
| Ph1 p 5a | FAEGLSGEPKGAAESSSKAA | 79-98 | HLA-DRB1*1101 SEQ ID NO: 27 |

### Example 6

### Ara h 1 Epitopes in Peanut Allergic Individuals

Tetramer Guided Epitope Mapping (TGEM) was used to identify the antigenic peptides within the peanut allergen Ara h 1 (Arachis hypogaea 1). Subsequently, HLA class II/Ara h 1-specific tetramers were used to determine the frequency and phenotype of Ara h 1-reactive T cells in peanut-allergic subjects. Cytokine profiles of Ara h 1-reactive T cells were also determined.

Multiple Ara h 1 epitopes with defined HLA restriction were identified. Ara h 1-specific CD4+ T cells were detected in all of the peanut-allergic subjects tested. Ara h 1-reactive T cells in allergic subjects expressed CCR4 but did not express CRTH2. The percentage of Ara h 1-reactive cells that expressed the β7 integrin was low compared to total CD4+ T cells. Ara h 1-reactive cells that secreted IFN-γ, IL-4, IL-5, IL-10 and IL-17 were detected.

In peanut-allergic individuals, Ara h 1-reactive T cells occurred at moderate frequencies, were predominantly CCR4+ memory cells and produced IL-4. Class II tetramers can be readily used to detect Ara h 1-reactive T cells in the peripheral blood of peanut allergic subjects without in vitro expansion and would be effective for tracking peanut-reactive CD4+ T cells during immunotherapy.

Peanut allergy is relatively common, affecting approximately 1% of children in the U.S. and the UK, and poses a significant risk of fatality. Peanut sensitivity typically presents early in life, but is often permanent, as only 20% of young children resolve their food allergy by adulthood. Currently, the only standard treatment options for peanut allergy sufferers are vigilant avoidance and administration of epinephrine in the event of an accidental ingestion. However, multiple approaches aimed to induce tolerance to peanut have been carried out.

Studies initiated more than 15 years ago demonstrated that desensitization is possible for peanut allergy by injection of crude peanut extract (Oppenheimer et al., Treatment of peanut allergy with rush immunotherapy. J Allergy Clin Immunol 1992; 90(2):256-62; Nelson et al., Treatment of anaphylactic sensitivity to peanuts by immunotherapy with injections of aqueous peanut extract. J Allergy Clin Immunol 1997; 99(6 Pt 1):744-51). However these injections were associated with frequent episodes of anaphylaxis (Bock et al., Further fatalities caused by anaphylactic reactions to food, 2001-2006. J Allergy Clin Immunol 2007; 119(4):1016-8.). More recently, peanut oral immunotherapy trials were attempted, and the outcomes were encouraging (Hofmann et al., Safety of a peanut oral immunotherapy protocol in children with peanut allergy. J Allergy 354 Clin Immunol 2009; 124(2):286-91, 291. 355; Jones et al., Clinical efficacy and immune regulation with peanut oral immunotherapy. J Allergy Clin Immunol 2009; 357 124(2):292-300, 300; Blumchen et al., Oral peanut immunotherapy in children with peanut anaphylaxis. J Allergy Clin Immunol 360 2010; 126(1):83-91). However, even in these recent oral immunotherapy trials, treatment was associated with side effects that required medication.

Allergic subjects with a documented record of peanut anaphylaxis and positive ImmunoCap scores for peanut-specific IgE were recruited. A subset of these subjects also had documented seasonal allergy. Atopic subjects without peanut allergy and non-atopic subjects with no clinical symptoms of allergy and negative ImmunoCap scores for Timothy grass pollen, cat, dust mite and peanut were also recruited. DNA samples were HLA typed using Dynal Unitray SSP Kits (Invitrogen) according to manufacturer's instructions.

Biotinylated HLA-DR proteins were purified. A total of 77 peptides (p1 to p77), which were 20 amino acids in length with a 12 amino acid overlap spanning the entire Ara h 1 sequence (including the signal peptide), were synthesized (Mimotopes, Clayton Australia). These peptides were divided into 14 pools of 5 peptides each plus a 15th pool of 7 peptides. These peptide mixtures were loaded into the biotinylated HLA-DR proteins to generate pooled tetramers. Cells were cultured for 14 days and then stained with pooled peptide tetramers. Cells from wells which gave positive staining were stained again using tetramers loaded with each individual peptide from the positive pool.

The frequency of Ara h 1-specific T cells was measured as described above. Briefly, 30 million PBMC in 200 μl of T cell culture medium were stained with 20 μg/ml PE-labeled tetramers at room temperature for 100 min. Cells were then stained with anti-CD3 FITC (eBioscience), anti-CD4 APC (eBioscience), anti-CD14 PerCP (BD Pharmingen) and anti-CD19 PerCP (BD Pharmingen) for 20 minutes at 4° C. Cells were washed and incubated with anti-PE magnetic beads (Miltenyi Biotec) at 4° C. for 20 minutes, washed again, and a 1/100th fraction saved for analysis. The other fraction was passed through a magnetic column (Miltenyi Biotec). Bound, PE-labeled cells were flushed and collected. Cells in the bound and pre-column fractions were stained with Via-Probe (BD Bioscience) for 10 minutes before flow cytometry. Data were analyzed using FlowJo (Tree Star), gating on FSC/SSC and excluding CD14+ and CD19+ populations and Via-Probe+ (dead) cells.

For phenotyping studies, antibodies against markers of interest were used instead of anti-CD3. Staining for Aln g 1 and Phl p 1 reactive T cells was carried out with DR0701/Aln g 1₁₃₇₋₁₅₆, DR1501/Aln g 1₁₃₇₋₁₅₆ and DR0401/Phl p 1₁₂₀₋₁₃₉ tetramers.

For intracellular staining of IFN-γ, IL-4, IL-17, IL-5, and/or IL-10, PBMCs were stimulated for 2 weeks with specific peptide, and then stained with the corresponding PE-labeled tetramers for 60 minutes at 37° C. Cells were then re-stimulated with 50 ng/mL PMA and 1 μg/mL ionomycin in the presence of 10 μg/ml monensin in 1 ml of complete media for 6 hours at 37° C., 5% CO2. Following re-stimulation, cells were stained with anti-CD4 (BD Pharmingen), anti-CD3 (eBioscience), and a combination of anti-CD14 (BD Pharmingen), anti-CD19 (Dako) and ViViD reagent (Invitrogen) to exclude non-specific tetramer staining. After 30 minutes at room temperature, cells were fixed with fixation buffer (eBioscience), washed twice with a permeabilization buffer (eBioscience), and stained in 200 μl permeabilization buffer with various combinations of anti-IFN-γ, anti-IL-17, anti-IL-10 (all from Biolegend), anti-IL-4 (eBioscience) and anti-IL-5 (Miltenyi Biotec). After 30 minutes at 4° C., cells were washed and immediately analyzed by flow cytometry.

A total of twelve peanut-allergic subjects with a history of anaphylaxis to peanut were recruited for this study. Six non-atopic subjects and 5 atopic subjects without peanut allergy were also recruited as control subjects. The Tetramer Guided Epitope Mapping approach was used to identify CD4+ T cell epitopes for Ara h 1 in peanut-allergic subjects as described in the methods section. Ara h1₁₆₉₋₁₈₈, Ara h1₃₂₁₋₃₄₀, Ara h1₄₅₇₋₄₇₆ and Ara h1₄₆₅₋₄₈₄ were identified as DR1101-restricted T cell epitopes. In summary, a total of 20 epitopes were identified, restricted by DR0101, DR0301, DR0401, DR0404, DR1101, DR1401, DR1502 and DRB5. It is likely that pairs of consecutive peptides, such as Ara h1₄₅₇₋₄₇₆/Ara h1₄₆₅₋₄₈₄ (restricted by DR1101) and Ara h 1₃₂₁₋₃₄₀/Ara h 1₃₂₉₋₃₄₈ (restricted by DR1401) contain an identical minimum epitope. In using TGEM, we did not identify any Ara h 1 epitopes restricted by DR0701 and DR1501. However, each subject with a DR0701 or DR1501 haplotype recognized Ara h 1 epitopes restricted by another class II allele. Therefore it is possible that there are no DR1501 and DR0701 restricted Ara h1 epitopes. Alternatively, DR0701 and DR1501 restricted Ara h 1 specific T cells may be of low avidity, making them difficult to detect using tetramers.

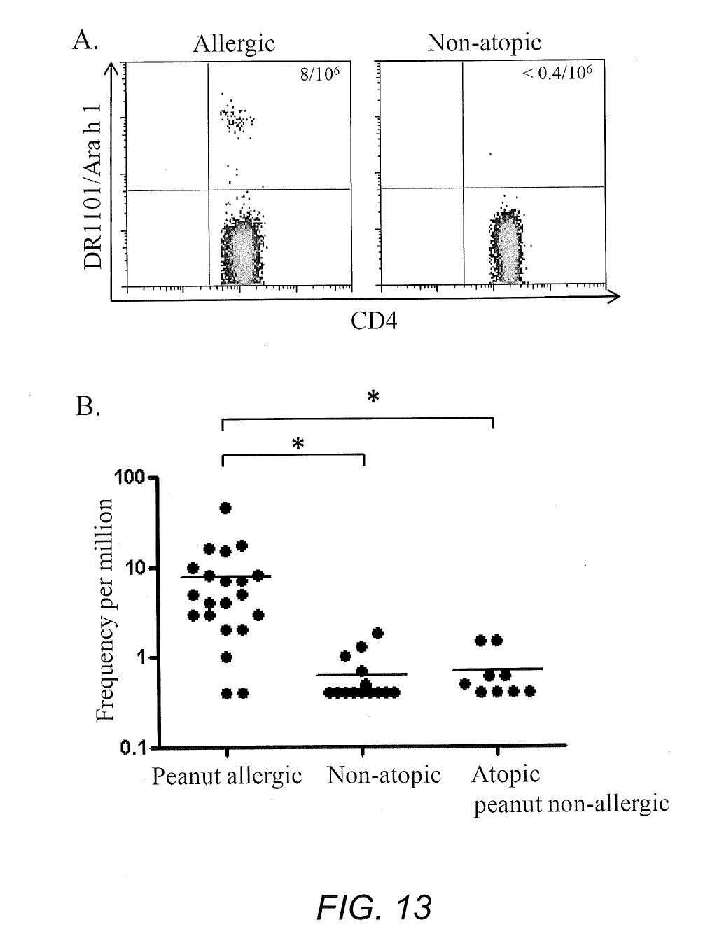

In order to detect Ara h 1-specific T cells directly ex vivo, anti-PE magnetic beads were used to enrich for PE-labeled tetramer-positive cells. This approach enabled the characterization of the phenotype and frequency of Ara h 1-reactive T cells in the peripheral blood without expanding PBMC in vitro. Representative results for a DR1101 peanut-allergic subject and a DR1101 non-atopic subject are shown in FIG. 13A. The results of additional experiments are summarized in FIG. 13B. The average frequency of Ara h 1-reactive T cells in peanut-allergic subjects was approximately 9 cells per million, while the average frequency in non-atopic subjects and atopic subjects without peanut allergy was less than 1 cell per million.

Ex vivo tetramer staining of Ara h 1-reactive T cells also allowed direct examination of cell surface phenotypes for Ara h 1-reactive T cells in allergic subjects, using surface markers such as CD45RA (a naïve T cell marker), CD45RO (a memory T cell marker), CRTh2 and CCR4 (Th2 markers), and CLA and β7 integrin (T cell homing markers). The expression of each of these markers on Ara h 1-reactive T cells was compared to that of total CD4+ T cells.

In total, the data indicated that Ara h 1-reactive T cells in peanut allergic subjects were memory T cells and expressed the Th2 marker CCR4. The majority of these cells did not express CRTh2, and only a small fraction of these cells expressed the gut homing marker δ7 integrin. The majority of Ara h 1-reactive T cells also expressed CD25. The frequency of Ara h 1-reactive T cells in non-allergic subjects was very low, which precluded the examination of their phenotypes. Three peanut-allergic subjects also had a seasonal allergy to Timothy grass or alder pollen. Since we had also developed appropriate tetramer reagents to study these pollen reactive T cells, we examined the phenotype of pollen-specific T cells in these peanut allergic subjects. This allowed a comparison of Ara h 1-reactive T cells with Aln g 1 (Alder pollen allergen) or Phl p 1 (Timothy grass pollen allergen) reactive T cells within the same subjects. The results of these experiments indicated that, while the majority of Ara h 1-, Aln g 1- and Phl p 1-reactive T cells expressed CCR4, only Aln g 1- and Phl p 1-205 reactive T cells expressed CRTh2.

The CCR4 surface phenotype of Ara h 1-reactive T cells indicated that these T cells belong to the Th2 linage. The Th2 phenotype of these cells was further confirmed by examining the cytokine profiles of Ara h 1-specific T cell lines and clones derived from peanut-allergic subjects. Ara h 1-specific cell lines were generated by stimulating the PBMC of peanut allergic subjects with antigenic Ara h 1 peptides for two weeks. Ara h 1-reactive T cell clones were isolated by sorting single Ara h 1 tetramer-positive T cells from Ara h 1 lines and subsequently expanding them with PHA. Cytokine profiles were examined by tetramer staining in conjunction with intra-cellular cytokine staining (ICS). It was observed that all of the Ara h 1-reactive cell lines and clones examined produced IL-4. At least one third of the lines also produced IL-5. More than half of the cell lines produced a low amount of IFN-γ. Multicolor ICS identified cell lines that produced IL-4 and IL-5 simultaneously or either IL-4 or IL-5 individually. Cell lines that produced IL-10 or IL-17 individually or in combination with IL-4 were also observed, though the percentage of IL-4 and IL-17 co-producers was minimal. Release of IL-5 and IL-13 by Ara h 1-specific lines was confirmed by measuring cytokine in the supernatants of our Ara h 1-stimulated T cell lines using the Meso Scale Discovery multiplex kit. These experiments indicated that T cell lines stimulated with Ara h 1 peptides produced at least 8 fold more IL-5 and IL-13 than IFN-γ. In total, these data indicated that the majority of Ara h 1-reactive CD4+ T cells in peanut-allergic subjects were Th2 cells, but also confirmed the existence of Ara h 1-228 reactive cells that produced IFN-γ, IL-10 and IL-17.

As previously mentioned, most of the Ara h 1-specific cells examined did not express CRTh2, the most definitive Th2 marker. However, CCR4 staining results and cytokine secretion profiles clearly indicated that these cells are functionally Th2-like. The fact that the subjects from this study have avoided contact with peanut allergens for a long period of time may have resulted in a lack of CRTh2 expression by Ara h 1-reactive T cells. This would suggest that occasional antigen stimulation is essential for the expression of CRTh2 by T cells. This notion is supported by our observation that 75% ( 6/8) of Ara h 1 specific T cell clones expressed various levels of CRTh2 after activation and subsequent resting (data not shown). However, it is also possible that these Ara h 1-specific cells could be CRTh2-negative for a functional reason and may play a role in the pathophysiology of allergy distinct from CRTh2-positive Th2 cells.

This study highlights the use of tetramer reagents to detect and characterize Ara h 1-reactive CD4+ T cells. Its findings demonstrate the feasibility of using Ara h 1-specific tetramers as a specific biomarker for monitoring peanut-specific CD4+ T cells in various settings. Although the frequency of Ara h 1-reactive CD4+ T cells is relatively low in peanut allergic subjects, there was a significant difference in the frequency of Ara h 1 CD4+ reactive T cells in allergic subjects compared with non-allergic subjects. More importantly, tetramer reagents can be used to monitor the surface phenotype of peanut reactive T cells. As such, immune monitoring with tetramers can provide unambiguous data regarding the function, frequency and phenotype of allergenic reactive T cells. These reagents, used independently or in conjunction with other assays, may open up new approaches to monitor changes in the phenotype and frequency of food allergen-reactive CD4+ T cells during immunotherapy.

TABLE 7
Ara h 1 CD4+ T cell epitopes

| | Ara h 1 | Amino Acid Sequence | |
|---|---|---|---|
| DRB1*0101 | Ara h 1 (201-220) | QRSRQFQNLQNHRIVQIEAK | SEQ ID NO: 28 |
| | Ara h 1 (233-252) | DNILVIQQGQATVTVANGNN | SEQ ID NO: 29 |
| | Ara h 1 (505-524) | KEGDVFIMPAAHPVAINASS | SEQ ID NO: 30 |
| DRB1*0301 | Ara h 1 (409-428) | NNFGKLFEVKPDKKNPQLQD | SEQ ID NO: 31 |
| DRB1*0401 | Ara h 1 (201-220) | QRSRQFQNLQNHRIVQIEAK | SEQ ID NO: 32 |
| | Ara h 1 (329-348) | FNEIRRVLLEENAGGEQEER | SEQ ID NO: 33 |
| | Ara h 1 (505-524) | KEGDVFIMPAAHPVAINASS | SEQ ID NO: 34 |
| | Ara h 1 (577-596) | QKESHFVSARPQSQSQSPSS | SEQ ID NO: 35 |
| DRB1*0404 | Ara h 1 (329-348) | FNEIRRVLLEENAGGEQEER | SEQ ID NO: 36 |
| | Ara h 1 (449-468) | NSKAMVIVVVNKGTGNLELV | SEQ ID NO: 37 |
| DRB1*1101 | Ara h 1 (169-188) | TSRNNPFYFPSRRFSTRYGN | SEQ ID NO: 38 |
| | Ara h 1 (321-340) | LEAAFNAEFNEIRRVLLEEN | SEQ ID NO: 39 |
| | Ara h 1 (457-476) | VVNKGTGNLELVAVRKEQQQ | SEQ ID NO: 40 |
| | Ara h 1 (465-484) | LELVAVRKEQQQRGRREEEE | SEQ ID NO: 41 |
| DRB1*1401 | Ara h 1 (321-340) | LEAAFNAEFNEIRRVLLEEN | SEQ ID NO: 42 |
| | Ara h 1 (329-348) | FNEIRRVLLEENAGGEQEER | SEQ ID NO: 43 |
| DRB1*1502 | Ara h 1 (201-220) | QRSRQFQNLQNHRIVQIEAK | SEQ ID NO: 44 |
| DRB5 | Ara h 1 (209-228) | LQNHRIVQIEAKPNTLVLPK | SEQ ID NO: 45 |
| | Ara h 1 (369-388) | SKEHVEELTKHAKSVSKKGS | SEQ ID NO: 46 |
| | Ara h 1 (489-508) | EEEGSNREVRRYTARLKEGD | SEQ ID NO: 47 |

TABLE 8
Cytokine Profiles of Ara h1 clones and lines

| HLA | | | | | | | |
|---|---|---|---|---|---|---|---|
| restric- | | Cell line/ | | | | | |
| tion | epitope | Clone | IFN-γ | IL-4 | IL-5 | IL-10 | IL-17 |
| DR0101 | Ara h 1₂₀₁₋₂₂₀ | Line 1 | − | ++ | −/+ | − | − |
| DR0101 | Ara h 1₂₀₁₋₂₂₀ | Line 2 | − | ++ | ND | ND | − |
| DR0401 | Ara h 1₂₀₁₋₂₂₀ | Clone 9 | − | ++ | ++ | − | − |
| DR0401 | Ara h 1₃₂₉₋₃₄₈ | Clone 3 | −/+ | ++ | ++ | − | − |
| DR0401 | Ara h 1₃₂₉₋₃₄₈ | Clone 7 | − | ++ | − | − | − |
| DR0401 | Ara h 1₅₇₇₋₅₉₆ | Clone 19 | − | ++ | ++ | − | − |
| DR0401 | Ara h 1₃₂₉₋₃₄₈ | Line 1 | + | ND | ND | ++ | − |
| DR0401 | Ara h 1₃₂₉₋₃₄₈ | Line 2 | + | ++ | − | + | − |
| DR0401 | Ara h 1₃₂₉₋₃₄₈ | Line 3 | −/+ | ++ | − | ++ | − |
| DR0404 | Ara h 1₃₂₉₋₃₄₈ | Line 1 | − | ++ | − | − | − |
| DR0404 | Ara h 1₃₂₉₋₃₄₈ | Line 2 | −/+ | ++ | −/+ | − | ++ |
| DR1101 | Ara h 1₁₆₉₋₁₈₈ | Line 1 | − | ++ | ++ | − | − |
| DR1101 | Ara h 1₁₆₉₋₁₈₈ | Line 2 | −/+ | ++ | ++ | − | − |
| DR1101 | Ara h 1₁₆₉₋₁₈₈ | Line 3 | + | ++ | ++ | + | − |
| DR1101 | Ara h 1₃₂₁₋₃₄₀ | Line 1 | −/+ | ++ | ++ | −/+ | − |
| DR1401 | Ara h 1₃₂₁₋₃₄₀ | Line 1 | ++ | + | −/+ | − | − |

<5% positive in ICS staining is −
6% to 10% is −/+
11% to 30% is +
Greater than 30% is ++

The foregoing is illustrative of the present invention, and is not to be construed as limiting thereof. The invention is defined by the following claims, with equivalents of the claims to be included therein.

1. A method of treating an allergic disorder in a subject in need thereof, comprising: administering one or more CD4+ T cell Th2 response eliciting polypeptides to said subject, to thereby treat said subject for said allergic disorder.

2. The method of claim 1, wherein said allergic disorder is seasonal rhinoconjuctivitis, animal dander allergy, food allergy, or venom anaphylaxis.

3. The method of claim 1, wherein said allergic disorder is an allergy to timothy grass pollen and said polypeptides are selected from the group consisting of: SEQ ID NO:1, SEQ ID NO:2, SEQ ID NO:3, SEQ ID NO:4, SEQ ID NO:5, SEQ ID NO:22, SEQ ID NO:23, SEQ ID NO:24, SEQ ID NO:25, SEQ ID NO:26, SEQ ID NO:27, and homologues thereof.

4. The method of claim 1, wherein said allergic disorder is an allergy to alder pollen and said polypeptides are selected from the group consisting of: SEQ ID NO:9, SEQ ID NO:10, SEQ ID SEQ ID NO:12, SEQ ID NO:13, SEQ ID NO:14, and homologues thereof.

5. The method of claim 1, wherein said allergic disorder is a feline allergy and said polypeptides are selected from the group consisting of: SEQ ID NO:16, SEQ ID NO:17, SEQ ID NO:18, SEQ ID NO:19, SEQ ID NO:20, SEQ ID NO:21, and homologues thereof.

6. The method of claim 1, wherein said allergic disorder is a peanut allergy and said polypeptides are selected from the group consisting of: SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, and homologues thereof.

7. The method of claim 1, further comprising determining the MHC class II genotype of said subject, and wherein said polypeptides are selected based upon the MHC class II genotype of said subject.

8. The method of claim 7, wherein said determining step comprises nucleic acid amplification.

9. The method of claim 1, wherein said subject is a human subject.

10.-12. (canceled)

13. A composition consisting essentially of polypeptides which elicit a Th2 CD4+ T cell response.

14. The composition of claim 13 further comprising a carrier.

15. The composition of claim 14 formulated for subcutaneous or sublingual administration.

16. The composition of claim 13, wherein said composition comprises:
(a) a polypeptide selected from the group consisting of: SEQ ID NO:1, SEQ ID NO:2, SEQ ID NO:3 SEQ ID NO:4, SEQ ID NO:5, SEQ ID NO:22, SEQ ID NO:23, SEQ ID NO:24, SEQ ID NO:25, SEQ ID NO:26, SEQ ID NO:27, and homologues thereof;
(b) a polypeptide selected from the group consisting of: SEQ ID NO:9, SEQ ID NO:10, SEQ ID SEQ ID NO:12, SEQ ID NO:13, SEQ ID NO:14, and homologues thereof;
(c) a polypeptide selected from the group consisting of: SEQ ID NO:16, SEQ ID NO:17, SEQ ID NO:18, SEQ ID NO:19, SEQ ID NO:20, SEQ ID NO:21, and homologues thereof; or
(d) a polypeptide selected from the group consisting of: SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, and homologues thereof.

17.-23. (canceled)

24. A method for testing or monitoring a CD4+ T cell response in a subject to an antigen, said antigen comprising a predetermined epitope, said method comprising:
obtaining a blood sample from said subject, said blood sample comprising CD4+ T cells;
providing a complex comprising said predetermined epitope and a predetermined MHC class II molecule that binds to said predetermined epitope;
contacting said CD4+ T cells with said complex; and
determining whether the response of said CD4+ T cells is a Th1 or a Th2 response;
to thereby determine the type of CD4+ T cell response to said antigen.

25. The method of claim 24, wherein said determining comprises detecting cytokine secretion and/or cell surface markers of said CD4+ T cells.

26. The method of claim 24, wherein said predetermined complex comprises MHC class II tetramers.

27. The method of claim 24, wherein said blood sample is a whole blood sample or a peripheral blood mononuclear cell (PBMC) sample.

28. The method of claim 27, wherein said blood sample has a volume of from 0.1 to 10 milliliters.

29. (canceled)

30. The method of claim 24, wherein said subject is afflicted with an allergic disease, an autoimmune disease, or a cancer.

31. The method of claim 24, wherein said antigen is a vaccine antigen.
